(19) United States
(12) Patent Application Publication
(10) Pub. No.: US 2026/0306526 A1
(43) Pub. Date: Oct. 8, 2026

(54) COMBINATION THERAPY FOR BACTERIAL INFECTIONS COMPRISING ZINC-CHELATOR, BETA-LACTAM ANTIBIOTIC AND SERINE BETA-LACTAMASE INHIBITOR

(71) Applicant: ADJUTEC PHARMA AS, Oslo (NO)

(72) Inventors: Pål RONGVED, Oslo (NO); Bjørn KLEM, Oslo (NO)

(21) Appl. No.: 19/481,617

(22) PCT Filed: May 9, 2024
(86) PCT No.: PCT/EP2024/062884

§ 371 Date: Nov. 5, 2025

(30) Foreign Application Priority Data
May 9, 2023 (GB) 2306833.1

(51) Int. Cl. A61K 31/706; A61K 31/407; A61K 31/427; A61K 31/43; A61K 31/431; A61K 31/439; A61K 31/444; A61K 31/551; A61P 31/04

(52) U.S. Cl. CPC A61K 31/706; A61K 31/407; A61K 31/427; A61K 31/43; A61K 31/431; A61K 31/439; A61K 31/444; A61K 31/551; A61P 31/04

(57) ABSTRACT

The invention provides a method of treatment of a bacterial infection which comprises co-administration of an effective amount of each of the following agents to a subject in need thereof: (A) a selective zinc-chelator which comprises one or more lipophilic, zinc chelating moieties covalently bound to one or more hydrophilic moieties, wherein said zinc chelating moieties are selective for Zn²⁺ ions and wherein said hydrophilic moieties are selected from hydrophilic monomeric, oligomeric and polymeric groups; (B) a p-lactam antibiotic selected from the group consisting of penicillins, monobactams and carbapenems; and (C) a serine β-lactamase inhibitor. Pharmaceutical compositions and kits which comprise a combination of agents (A), (B) and (C) are so provided.

### TECHNICAL FIELD

The present invention relates to improvements in and relating to the treatment of bacterial infections and bacterial biofilms that harbour such infections.

In particular, the invention relates to a combination therapy for use in such treatment which comprises co-administration of known adjuvants and antibacterial agents. The invention further relates to novel pharmaceutical compositions comprising the adjuvants and antibacterial agents, to kits that contain these, and to their use in such treatment.

### BACKGROUND OF THE INVENTION

The global increase in antimicrobial resistance is currently undermining our ability to treat bacterial infections and has become a critical public health threat worldwide. A cornerstone in treatment of serious and life-threatening infections caused by multidrug-resistant (MDR) gram-negative bacterial pathogens such as Klebsiella pneumoniae and Escherichia coli has been the carbapenem β-lactam antibiotics, e.g. penicillins and carbapenems.

The major advantage of carbapenems has been their relative stability towards β-lactamases, such as the extended-spectrum β-lactamases (ESBLs) and AmpCs, which constitute common resistance mechanisms against β-lactams (Bush K., J Infect. Chemother. (2013), 19(4): 549-59). However, we now observe a global increase in dissemination and diversity of β-lactamases (carbapenemases) with the ability to inactivate carbapenems (Logan, L. K. et al., J. Infect Dis. (2017), 215 (Suppl 1):S28-S36).

The impact of carbapenem-resistance is further illustrated in a European study in which carbapenem-resistance was shown to be the major contributor to the burden of infections by antibiotic-resistant bacteria in many countries (Cassini A. et al., Lancet Infectious diseases (2019), 19, 56-66). Moreover, a common feature of carbapenemase-producing gram-negative bacteria is MDR (multi-drug resistance), including resistance towards non-β-lactam antimicrobials, resulting in severely limited treatment options (Perez F, Expert Opin. Pharmacother. (2016), 17(6), 761-81).

An especially important disease area in which novel, more efficient bacterial enzymes play a vital role, is that of resistant microbes. Infectious diseases are a leading cause of death worldwide and account for millions of deaths annually including nearly two-thirds of all childhood mortality at less than 5 years of age. There is serious concern regarding new and re-emerging infectious diseases for which effective therapies are lacking (World Health Organization reports 2020 and 2021). The introduction of new, more potent derivatives of existing antibiotics provides only temporary solutions since existing resistance mechanisms rapidly adapt to accommodate the new derivatives (Theuretzbacher U. Curr. Opin. Pharmacol. (2011), 11, 433-438).

Although resistant gram-positive bacteria pose a significant threat, the emergence of multi-drug resistant (MDR) strains of common gram-negative pathogens such as Escherichia coli are of special concern. Pan-resistance or extreme drug resistance are now commonly used terms to describe clinically important isolates of Pseudomonas aeruginosa, Acinetobacter baumannii and Enterobacteriaceae that are resistant to virtually all antibiotics (Bush K. et al., Clin. Microbiology Rev. (2020), 33, e00047-19).

There are few, if any, antimicrobial agents effective against gram-negative bacteria either in or entering phase I clinical trials that will address this critical need (Butler M S. et al., J. Antibiotics (2013), 66, 571-591). One important feature of bacteria, especially gram-negative bacteria, is that they have two cell membranes, one outer membrane that is more permeable and one inner cell membrane resembling an eucaryotic cell membrane. One important group of enzymes involved in antimicrobial resistance is the β-lactamases (Bush K. et al., Annu. Rev. Microbiol. (2011), 65, 455-478). They are excreted into the volume between these membranes (i.e. the periplasmic space) which is more accessible to drugs. The β-lactamases are divided into two main families and four classes, the serine β-lactamases (SBLs) and the metallo-β-lactamases (MBLs). The SBLs are classified as Ambler classes A, C and D. Examples of SBLs in class A are CepA, KPC-2, IMI-1, SME-1, PC1, TEM-1, TEM-2, TEM-3, TEM-30, TEM-50, SHV-1, SHV-2, SHV-10, CTX-M-15, PER-1, VEB-1, PSE-1, CARB-3, and RTG-4. SBLs in Ambler class C include AmpC, CMY-1, ACT-1, FOX-1, MIR-1, GC1, CMY-10, CMY-19, and CMY-37, and those in Ambler class D include OXA-1, OXA-10, OXA-11, OXA-15, OXA-23, and OXA-48. The MBLs are classified in Ambler class B and include IMP, VIM, SPM, IND, NDM, DIM, GIM, SIM, AIM, CAU-1, GOB-1, FEZ-1, CcrA, IND-1, L1, CphA, Sfh-1, and ImiS.

The main distinction between SBLs and MBLs is that SBLs possess an active site serine hydroxy group, while MBLs require the presence of metal ions for activity. β-lactamases with carbapenemase activity have been identified in both of these families including SBLs such as KPC and OXA-48, and MBLs such as NDM, VIM and IMP. The recent introduction of serine carbapenemase inhibitors such as avibactam and vaborbactam used in combination with β-lactams has provided treatment options against serine carbapenemase-producing gram-negative pathogens.

Other SBL inhibitors with inhibitory activity against carbapenemases, such as relebactam and ETX2514, are either in late-stage development or early phase I clinical trials. However, none of these β-lactamase inhibitors possess inhibitory activity against MBLs. Despite several reports of promising MBL inhibitors, including aspergillomarasmine A, dipicolinic acid derivatives, ANT431, bisthiazolodines, and bismuth antimicrobials, no selective and efficient inhibitors are close to market. Consequently, new treatment options for infections caused by MBL-producing gram-negatives, including NDM-producing Enterobacteriales, are urgently required.

The recent introduction of serine carbapenemase inhibitors such as avibactam, vaborbactam and relebactam used in combination with β-lactams provides treatment options against serine carbapenemase-producing gram-negative pathogens (Zhanel G. G., et al., Drugs (2018), 78, 65-98; H. Wright, H. et al, European Society of Clinical Microbiology and Infectious Diseases (2017), 23, 704-712). Unfortunately, none of these β-lactamase inhibitors possess inhibitory activity against MBLs.

The recent Italian outbreak of NDM-producing Enterobacteriaceae is significant due not only to its size but also the change in epidemiology of carbapenem-resistant Enterobacteriaceae (CRE) from endemic KPC-producing CRE to NDM-producing CRE and the subsequent reduction in treatment options (ECDC Report, Italian Outbreak NDM, 2018.2019, Stockholm). Consequently, new treatment options for infections caused by MBL-producing gram-negatives, including NDM-producing Enterobacteriales, are urgently required.

Possible treatment options include cefiderocol (Zhanel, 2018) and the combination aztreonam-avibactam (Chew K. L. et al., Antimicrob. Agents Chemother. (2018), 62, e00414-18). Combinations of β-lactams and β-lactam enhancers such as zidebactam (Moya B., Antimicrob. Agents Chemother. (2019), 597, 63: e00128-19) have also shown promising activity. Moreover, several MBL inhibitors, including aspergillomarasmine A (King A. M., Nature. (2014), 510, 503-6), dipicolinic acid derivatives (Chen A. Y. et al, J. Med Chem (2017), 60(17): 7267-83), ANT431 (Everett M. et al., Antimicrob. Agents Chemother. (2018), 62: e00074-18), bisthiazolodines (Hinchliffe P, et al., Proc. Natl. Acad. Sci. USA (2016), 113(26), E3745-54) and bismuth antimicrobials (Wang R. et al., Nature Commun. (2018), 9(1): 439) have been reported. Recently, VNRX-5133 (taniborbactam), a dual SBL and MBL inhibitor has shown potent activity in combination with cefepime against MBL-producers (Hamrick J. C. et al., Antimicrob. Agents Chemother. (2020), 64, e01963-19). However, taniborbactam in combination with cefepime, for example, has some weaknesses in having no effect against the MBL class IMP.

Thus, no selective and efficient MBL inhibitors are approved for clinical use (Bush K. et al., Clin. Microbiology Rev. (2020), 33, e00047-19). Metallo-β-lactamases (MBLs) belong to a large group of proteins only found in bacteria and, like penicillin-binding proteins (PBPs), have the ability to interact with β-lactams. Examples of PBPs and enzymes that bind β-lactams are MBLs, serine β-lactamase-like protein (LACTB), D,D-transferase, D-Ala (D,D)-carboxypeptidase, the D-Alanyl-D-alanine Dipeptidases VanA, VanX, VanY and others, as reviewed by Sauvage E. et al. in FEMS Microbiol. Rev. (2008), 32, 234-258. This class of proteins is only found in bacterial biology. Examples of compounds having affinity for PBPs are β-lactam antibiotics. β-Lactams have been the historical anchor of antibacterial chemotherapy and include penicillins, cephalosporins, monobactams and carbapenems (Bush K et al., Annu. Rev. Microbiol. (2011), 65, 455-478). MBLs are emerging as one of the most clinically important family of β-lactamases (Patel et al., Front. Microbiol. (2013), 4, 48, Walsh et al., Int. J. Antimicrob. Agents (2010), S8-S14). The clinically most important MBLs, the IMP-, VIM-, GIM- and NDM-groups, are now widespread in a variety of gram-negative species. In particular, VIM- and NDM-enzymes have emerged as the dominant MBLs. The unprecedented global dissemination of NDM highlights the enormity of the problem. Since the first report in 2008, NDM has been identified in Australia, Africa, North America, Asia and many European countries (Johnson A. P. et al., J. Med. Microbiol. (2013), 62, 499-513). Worryingly, NDM is found in numerous gram-negative species and in the environment (Walsh T. R. et al., Lancet Infect. Dis. (2011), 11, 355-362).

Successful inhibitors of class A serine β-lactamases are clinically available, but lack inhibitory activity against MBLs (Drawz S. M. et al., Clin. Microbiol. Rev. (2010), 23, 160201). Inspired by the commercial success of the paradigm Augmentin (clavulanic acid—a suicide substrate for serine β-lactamases—and amoxicillin) several research groups have focused on similar approaches to develop inhibitors, but as yet no molecules that combine potency with activity against multiple MBL targets have reached clinical trials (Drawz S. M. et al., Antimicrob. Agents Chemother. 2014).

For the three clinically most threatening MBLs—the IMP, NDM and VIM groups—most inhibitors are reported for IMP-1, while few inhibitors are found for VIM-2 and NDM. For NDM, a natural fungal product, aspergillomarasmine A, has been identified as an MBL inhibitor and shown in vivo activity in mouse models (King A. M. et al., Nature (2014), 510, 503-506). However, relatively high doses of aspergillomarasmine A are required to reverse carbapenem resistance. Other therapeutic options include the use of tri-β-lactam therapy incorporating a monobactam (Martinez, Future Med. Chem. (2012), 4(3), 347-59); however, the MICs are not impressive, and in vivo activity is severely compromised by the bacterial inoculum (Page et al., Antimicrob. Agents Chemother. (2011), 66, 867-73).

Whereas in MBLs, where formation of a non-covalent reactive complex with the β-lactam is facilitated by Zn²⁺, distinct mechanisms exist for SBLs such as KPC and OXA-48, that utilise an active-site serine for hydrolysis. The serine-lactamases function by initially forming a covalently acylated enzyme. To date, a clinically available compound that inhibits both SBLs and MBLs has not yet been introduced.

An urgent medical challenge rising today is the increasing co-existence of MBLs and SBLs constitutively (constantly) expressed in the same pathogen. Thus, there is a need for inhibitors that simultaneously address MBLs and SBLs and which are capable of inhibiting a wide range of MBL isotypes (e.g. NDM, VIM and IMP) and at the same time SBLs (e.g. KPC, OXA and P99/AmpC). However, developing such ultrabroad-spectrum inhibitors is highly challenging because MBLs and SBLs possess markedly different mechanisms of action in inactivating β-lactam antibiotics. The variety in structural topologies of these enzymes and their markedly different mechanisms of action makes it challenging to develop an inhibitor capable of tackling a wide range of different bacterial enzymes.

Important β-lactam antibiotics are the penicillins, cephalosporin and carbapenem classes. Many compounds have been reported as having MBL-inhibiting activities. In bacteria, zinc sensing is carried out by regulators of different families, including SmtB/ArsR, MerR, TetR, MarR, and the Fur family (Napolitano et al., Journal of Bacteriology (2012), 2426-2436). In some cases, the MBL inhibitors contain a Zn²⁺-binding group that can interact strongly with the central metal ion(s). Compounds which bind metal ions, so-called metal “chelators”, have been shown to affect bacterial biological mechanisms.

Biofilm formation is an important bacterial resistance mechanism that contributes to the growing therapeutic concern globally. Biofilms consist of extracellular polymeric substances (EPS). These are natural hydrophilic carbohydrate polymers of high molecular weight secreted by microorganisms into their environment, and determine the physiochemical properties of a biofilm. Biofilm formation with the nocosomial infections produced by multidrug-resistant gram-negative species producing metallo-β-lactamases, e.g. Acinetobacter baumannii, Pseudomonas aeruginosa, Klebsiella pneumoniae and E. coli, is problematic since they affect all traditional activity at hospitals, like surgery and wound healing. Of particular concern is MBL-producing Acinetobacter baumannii, as described by Peymani et al., Jpn. J. Infect. Dis. (2011), 64, 69-71. MBL-production was also found to be significantly higher in biofilm-positive isolates of Pseudomonas aeruginosa, as described by Heydari et al., Jundishapur J Microbiol. (2015), 8(3), e15514. Thus, there is a medical need for new drugs with acceptable toxicity and selectivity that are active against gram-negative bacteria harbouring MBL.

Zinc chelators have been suggested as antibacterial agents, e.g. in WO 2009/140215, as inhibitors of biofilm formation, e.g. in WO 2011/63394 and WO 2009/155088, or as antiviral agents, e.g. in WO 2004/71425 and WO 2006/43153, but in these cases the compounds lack biological or metal-chelating selectivity or have high solubility in fat, high log P, and are therefore often toxic to eukaryotic cells. This lack of selectivity may lead to undesired toxicity of amino-polycarboxylate (APC) chelators and other biological effects when treating specific infections by a target organism in a host organism, e.g. when it is desirable to affect only specific microorganisms whilst a low toxicological effect on the host organism or other species (which are not a target for the treatment in question) is desired.

WO 2015/049546 and WO 2018/033719 describe new classes of selective zinc-chelating compounds. The chelator part of these compounds is highly selective for zinc (Zn²⁺ ions) and the compounds are made hydrophilic by attaching a side chain, for example a carbohydrate-like moiety, which reduces the toxicity of the compounds. The compounds described in WO 2018/033719 are selective inhibitors of metallo-β-lactamases with no intrinsic antibiotic effects. They are used as adjuvants together with β-lactam antibiotics of the carbapenem class, exemplified by meropenem (MEM). When meropenem is used alone, many clinically isolated gram-negative bacteria with MBL as a resistance mechanism are shown to be resistant to the antibiotic, for example Klebsiella pneumoniae, E. coli and Pseudomonas aeruginosa. When MEM is used in combination with the compound of Example 26 of WO 2018/033719 against the same strains, MEM shows inhibitory effect—i.e. the sensitivity of these bacteria towards the antibiotic drug is reinstated. However, this combination has poor effect towards certain strains of Pseudomonas aeruginosa and many strains of Acinetobacter baumannii, and has no effect against bacteria which rely on the other main class of bacterial resistance enzymes, namely the SBLs.

An ongoing need thus exists for alternative therapeutic treatments that are effective against bacterial infections, in particular against infections involving bacterial strains that have a defence mechanism to current treatments, such as those that rely on serine β-lactamase inhibitors (SBLs).

### SUMMARY OF THE INVENTION

It has now been found that the combined use of a selective zinc-chelator, a serine β-lactamase inhibitor and a β-lactam antibiotic in a single therapeutic regime provides significant advantages in the treatment of bacterial infections, in particular in the treatment of such infections that are associated with bacteria harbouring resistance enzymes, such as MBLs and SBLs.

The results provided herein evidence a ‘greater than expected’ effect associated with the combination of a selective zinc-chelator as disclosed in WO 2018/033719, avibactam (a serine β-lactamase inhibitor) and meropenem (a β-lactam antibiotic) against various bacterial strains harbouring resistance enzymes, such as Klebsiella pneumoniae Kpn St147 (known to be associated with the two constitutively expressed resistant mechanisms NDM-1 and KPC-2), Klebsiella pneumoniae ST101 (OXA-48), Klebsiella pneumoniae K66-45 (NDM-1) and Klebsiella pneumoniae BAA1705 (KPC-2). Specifically, it has surprisingly been found that when the selective zinc-chelator and meropenem are combined with avibactam, the effect on resistant bacteria is even greater than the combination of the selective zinc-chelator and the carbapenem. Furthermore, by mathematically analysing the microbiological test data, it has been found that the effect of the three agents together is not only additive, but synergistic, compared to the combination of the selective zinc-chelator and the carbapenem. This finding is beyond expectation and supports a novel approach to the treatment of bacterial infections as herein described in which all three agents are co-administered as part of the same therapeutic regime. Additional results provided herein evidence synergy for other combinations of a selective zinc-chelator, a serine β-lactamase inhibitor and a β-lactam antibiotic against a range of bacterial strains that harbour resistance enzymes.

This novel approach to the treatment of bacterial infections can be expected to extend to the combination of the following agents: (A) a selective zinc-chelator as defined herein; (B) a β-lactam antibiotic which is a penicillin, a monobactam or a carbapenem; and (C) a serine β-lactamase inhibitor. In addition to the treatment and/or prevention of bacterial infections, it is also proposed that this combination therapy may be used in the treatment or prevention of bacterial biofilms which harbour such infections, specifically in the disruption, removal or prevention of bacterial biofilms.

In one aspect the invention provides a method of treatment of a bacterial infection, said method comprising co-administration of an effective amount of each of the following agents to a subject in need thereof:
(A) a selective zinc-chelator which comprises one or more lipophilic, zinc chelating moieties covalently bound to one or more hydrophilic moieties, wherein said zinc chelating moieties are selective for Zn²⁺ ions and wherein said hydrophilic moieties are selected from hydrophilic monomeric, oligomeric and polymeric groups;
(B) a β-lactam antibiotic selected from the group consisting of penicillins, monobactams and carbapenems; and
(C) a serine β-lactamase inhibitor.

In another aspect the invention provides (A) a selective zinc-chelator as herein defined for use in the treatment of a bacterial infection in a subject by co-administration with:
(B) a β-lactam antibiotic selected from the group consisting of penicillins, monobactams and carbapenems; and
(C) a serine β-lactamase inhibitor.

In another aspect the invention provides (B) a β-lactam antibiotic selected from the group consisting of penicillins, monobactams and carbapenems for use in the treatment of a bacterial infection in a subject by co-administration with:
(A) a selective zinc-chelator as herein defined; and
(C) a serine β-lactamase inhibitor.

In another aspect the invention provides (C) a serine β-lactamase inhibitor for use in the treatment of a bacterial infection in a subject by co-administration with:
(A) a selective zinc-chelator as herein defined; and
(B) a β-lactam antibiotic selected from the group consisting of penicillins, monobactams and carbapenems.

In another aspect the invention provides the use of (A) a selective zinc-chelator as herein defined in the manufacture of a medicament for use in the treatment of a bacterial infection in a subject by co-administration with (B) β-lactam antibiotic selected from the group consisting of penicillins, monobactams and carbapenems; and (C) a serine β-lactamase inhibitor.

In another aspect the invention provides the use of (B) a β-lactam antibiotic selected from the group consisting of penicillins, monobactams and carbapenems in the manufacture of a medicament for use in the treatment of a bacterial infection in a subject by co-administration with (A) a selective zinc-chelator as herein defined; and (C) a serine β-lactamase inhibitor.

In another aspect the invention provides the use of (C) a serine β-lactamase inhibitor in the manufacture of a medicament for use in the treatment of a bacterial infection in a subject by co-administration with (A) a selective zinc-chelator as herein defined; and (B) a β-lactam antibiotic selected from the group consisting of penicillins, monobactams and carbapenems.

In another aspect the invention provides a pharmaceutical composition comprising:
(A) a selective zinc-chelator which comprises one or more lipophilic, zinc chelating moieties covalently bound to one or more hydrophilic moieties, wherein said zinc chelating moieties are selective for Zn²⁺ ions and wherein said hydrophilic moieties are selected from hydrophilic monomeric, oligomeric and polymeric groups;
(B) a β-lactam antibiotic selected from the group consisting of penicillins, monobactams and carbapenems;
(C) a serine β-lactamase inhibitor; and
(D) one or more pharmaceutically acceptable carriers or excipients.

In a further aspect the invention provides a kit comprising:
(i) a first container containing (A) a selective zinc-chelator which comprises one or more lipophilic, zinc chelating moieties covalently bound to one or more hydrophilic moieties, wherein said zinc chelating moieties are selective for Zn²⁺ ions and wherein said hydrophilic moieties are selected from hydrophilic monomeric, oligomeric and polymeric groups;
(ii) a second container containing (B) a β-lactam antibiotic selected from the group consisting of penicillins, monobactams and carbapenems;
(iii) a third container containing (C) a serine β-lactamase inhibitor; and
(iv) optionally instructions for carrying out a method of treatment of a bacterial infection in a subject.

### DETAILED DESCRIPTION OF THE INVENTION

The first agent (A) for use in the invention is a selective zinc-chelator. The selective zinc-chelator comprises one or more (e.g. one or two) lipophilic, zinc chelating moieties covalently bound to one or more (e.g. one, two or three) hydrophilic moieties, wherein said zinc chelating moieties are selective for Zn²⁺ ions and wherein said hydrophilic moieties are selected from hydrophilic monomeric, oligomeric and polymeric groups.

In some embodiments, the selective zinc-chelator for use in the invention is a compound having the general formula (I), or a stereoisomer, pharmaceutically acceptable salt or prodrug thereof:

$$Q - L - W \qquad ( I )$$

wherein:
Q is a lipophilic, zinc chelating moiety which is selective for Zn²⁺ ions;
L is a covalent bond or a linker; and
W is a hydrophilic monomeric, oligomeric or polymeric group, preferably a hydrophilic group comprising hydrogen bond donor and hydrogen bond acceptor atoms selected from H, N, O, S and P, e.g. a hydrophilic group comprising one or more functional groups selected from —OH, —SH, —CO₂H, —SO₃H, —PO₃H₂, —B(OH)₂, and aliphatic or aromatic nitrogen-containing groups.

In some embodiments, the chelating moiety Q in formula (I) is a lipophilic, zinc chelating moiety which is selective for Zn²⁺ ions and which comprises at least one, preferably two or more (e.g. 2, 3 or 4), optionally substituted, unsaturated heterocyclic rings, e.g. 5 or 6-membered heterocyclic rings (such rings preferably include at least one heteroatom selected from N, S and O, preferably N); wherein any optional substituents may be selected from C₁₋₆ alkyl, C₁₋₆ alkoxy, halogen, nitro, cyano, amine, and substituted amine.

In some embodiments, the chelating moiety Q in formula (I) comprises one or more optionally substituted heteroaryl groups, preferably two or more heteroaryl groups, e.g. such groups in which each heteroaryl ring has at least one nitrogen atom in the ring structure (e.g. pyridine, especially unsubstituted pyridine). In some embodiments, the chelating moiety Q in formula (I) is derived from picolinic acid and its derivatives (e.g. from picoylamine). In some embodiments, the chelating moiety Q comprises two or more (e.g. two, three or four) 2-pyridyl-methyl units.

Non-limiting examples of the chelating moiety Q in formula (I) include the following groups:

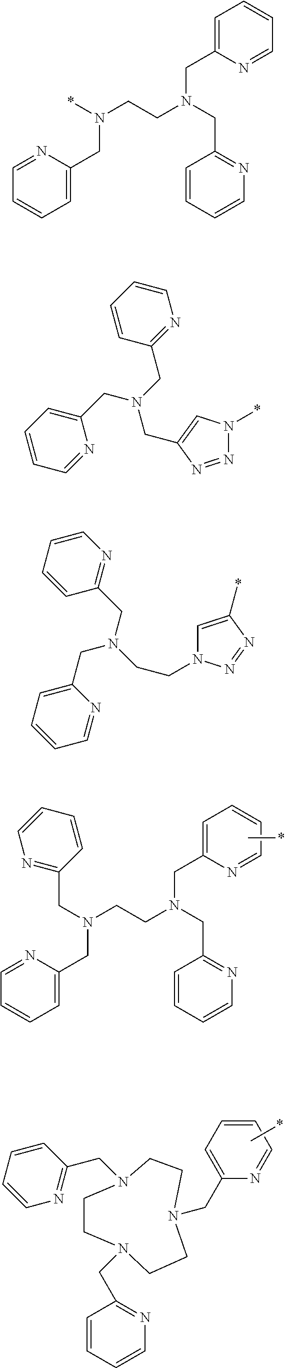

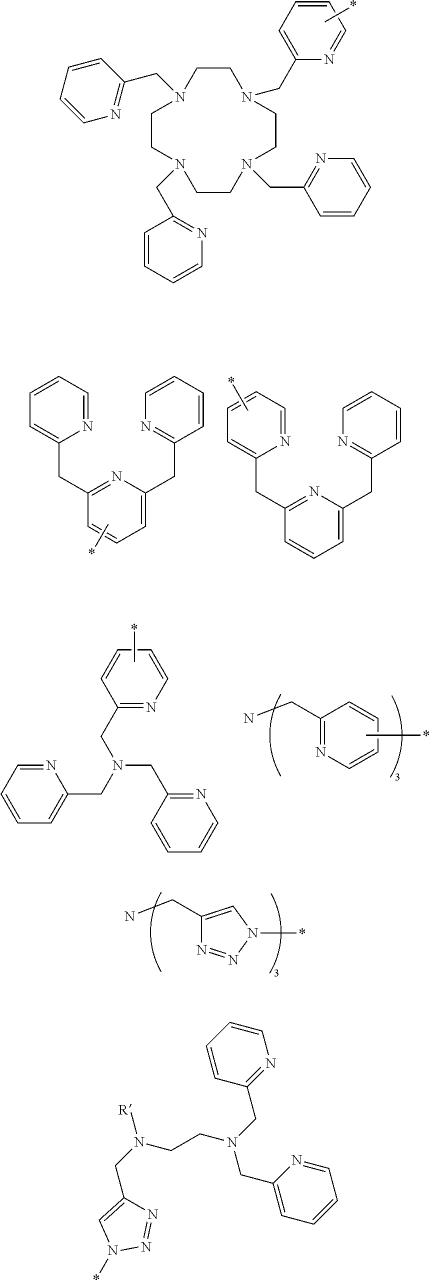

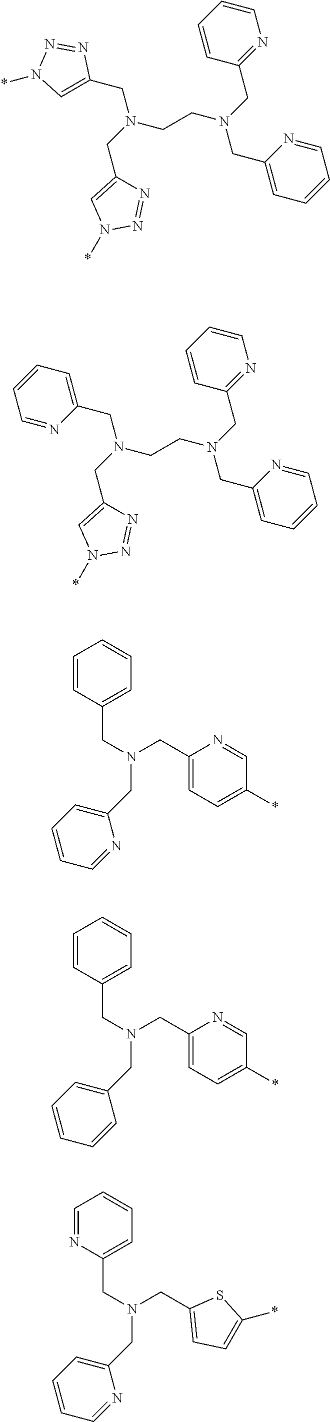

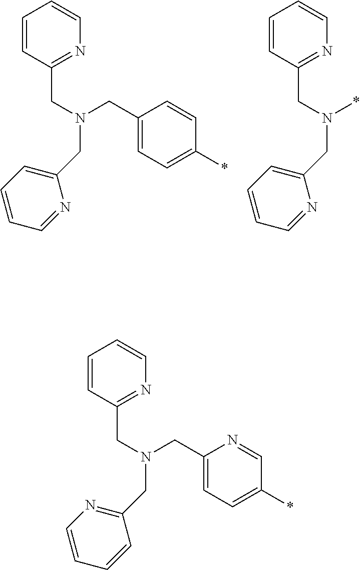

wherein * denotes the point (or points) of attachment of the chelating moiety to the remainder of the molecule, e.g. to a linker group L as herein defined; and R′, where present, is H or C₁₋₆ alkyl, e.g. C₁₋₃ alkyl, e.g. methyl.

In some embodiments, the linker L in formula (I) comprises a bond or an alkylene chain (preferably a C₁₋₈ alkylene, e.g. a C₁₋₆ alkylene) optionally substituted by one or more groups selected from C₁₋₃ alkyl, —O(C₁₋₃ alkyl), and —OR′ (where R′ is H or C₁₋₆ alkyl, preferably C₁₋₃ alkyl, e.g. methyl); and in which one or more —CH₂— groups (e.g. all —CH₂— groups) of the alkylene chain may be replaced by a group independently selected from —O—, —S—, —CO—, —NR″— (where R″ is H or C₁₋₆ alkyl, preferably C₁₋₃ alkyl, e.g. methyl), and an optionally substituted carbocyclic or heterocyclic ring (including monocyclic, bicyclic, tricyclic and fused rings). Any optional substituents may be selected from C₁₋₆ alkyl, C₁₋₆ alkoxy, halogen, nitro, cyano, amine, and substituted amine. In one embodiment, the linker may be interrupted by an optionally substituted aryl or heteroaryl ring, preferably an optionally substituted phenyl or triazole ring, e.g. an unsubstituted phenyl ring.

In some embodiments, the linker L comprises a bond, or a C₁₋₈ alkylene chain (preferably a C₁₋₆ alkylene chain, e.g. a C₁₋₃ alkylene chain) in which one or more —CH₂— groups (e.g. all —CH₂— groups) of the alkylene chain are optionally replaced by a group independently selected from —O—, —S—, —CO—, —NR″— (where R″ is independently H or C₁₋₆ alkyl, preferably C₁₋₃ alkyl, e.g. methyl), and an unsubstituted phenyl ring.

In some embodiments, the selective zinc-chelator for use in the invention is a compound of formula (II), or a stereoisomer, pharmaceutically acceptable salt or prodrug thereof:

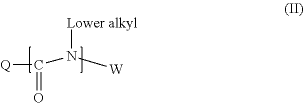

wherein Q and W are as herein defined; and
“lower alkyl” represents any straight-chained or branched C₁₋₆ alkyl group, preferably a C₁₋₄ alkyl group, e.g. a C₁₋₃ alkyl group such as methyl.

In formula (I) and formula (II), group W is a hydrophilic monomeric, oligomeric or polymeric group as herein defined.

In some embodiments, group W is a polyhydroxylated aliphatic or alicyclic group.

In some embodiments, group W comprises one or more of the following groups: a sugar moiety, a carboxylic acid or derivative thereof (e.g. an ester), an alcohol, an amine or substituted derivative thereof, and a boronic acid. The sugar moiety may be a mono-, di- or polysaccharide, an amino sugar, or a derivative thereof (e.g. an acetylated derivative). In one embodiment, the sugar moiety is a cyclic or acyclic monosaccharide. The alcohol can be a linear or branched, mono-, di- or tri-alcohol, e.g. a short-chain (e.g. C₁₋₆) linear or branched alcohol. The amine may be linear, branched or cyclic, preferably —NH₂, —NHR (where R is C₁₋₆ alkyl, e.g. C₁₋₃ alkyl), optionally substituted piperazine or morpholine (any optional substituents may be selected from —OH and C₁₋₃ alkyl, e.g. methyl). The boronic acid may be cyclic or acyclic and, optionally, the boronic acid group may form part of a 6-membered ring optionally substituted by one or more functional groups, e.g. carboxyl groups or derivatives thereof.

Examples of group W include, but are not limited to, the following groups:

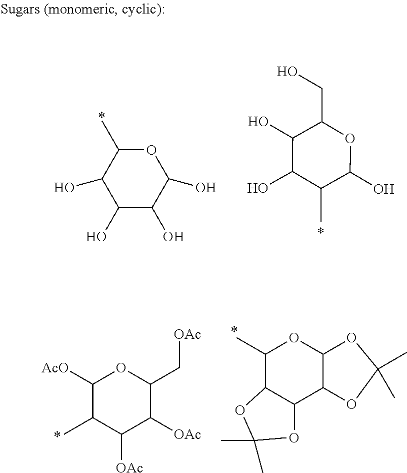

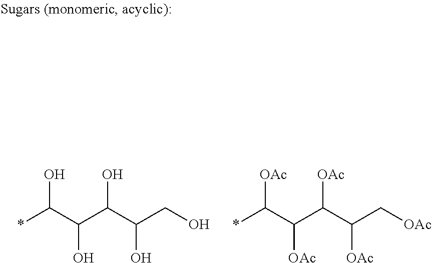

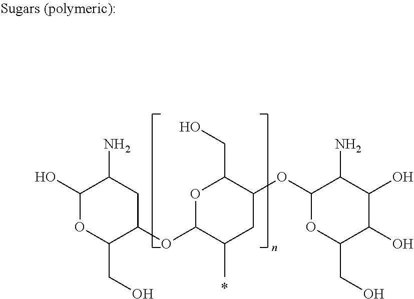

Carboxylic acids and Derivatives:
*—CO₂H
*—CO₂⁻
*—CO₂X (wherein X is a monovalent metal ion, e.g. Li⁺, Na⁺, K⁺ or C₁₋₆ alkyl, e.g. C₁₋₃ alkyl).

### Amines and Derivatives:

*—NH₂
*—NH₃⁺
*—NH₃⁺Y⁻ (wherein Y is Cl⁻, Br⁻, or I⁻)
*—NHR (where R is C₁₋₆ alkyl, e.g. C₁₋₃ alkyl, e.g. methyl)

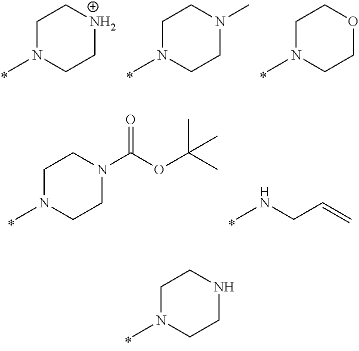

Preferred examples of such amines and their derivatives include:
*—NH₂
*—NH₃⁺
*—NHR (where R is C₁₋₆ alkyl, e.g. C₁₋₃ alkyl, e.g. methyl)

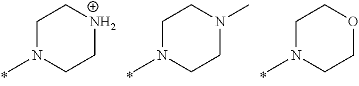

### Alcohols and Derivatives:

—OH
—OX (wherein X is a monovalent metal ion, e.g. Li⁺, Na⁺, K⁺ or C₁₋₆ alkyl, e.g. C₁₋₃ alkyl)
—OR (where R is C₁₋₆ alkyl, e.g. C₁₋₃ alkyl, e.g. methyl)

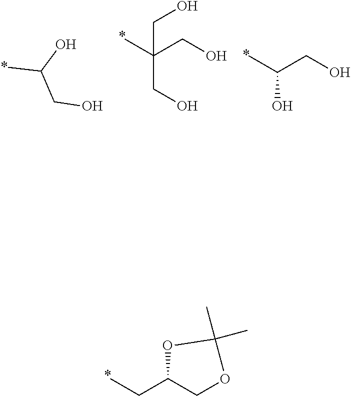

where * denotes the point of attachment of the hydrophilic group to the remainder of the molecule, e.g. to a linker group L as herein defined.

### Cyclic Boronic Acids:

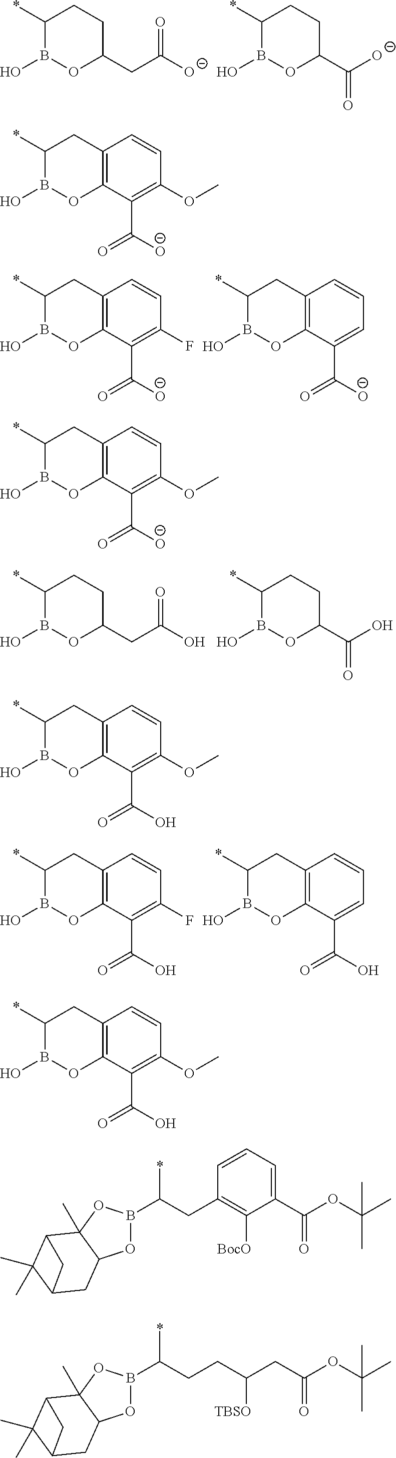

### Examples of Such Cyclic Boronic Acids Include the Following:

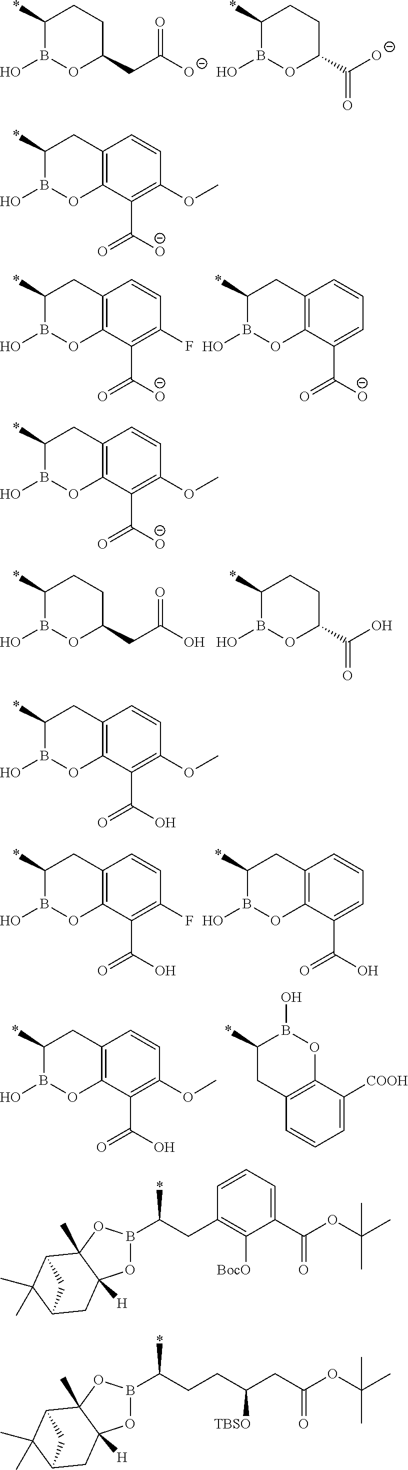

### Preferred Examples of Such Cyclic Boronic Acids Include:

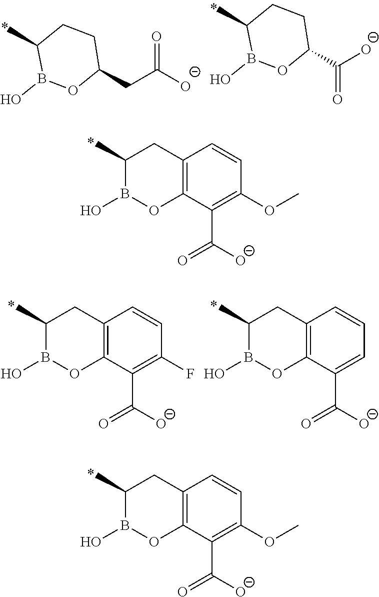

### Acyclic Boronic Acids:

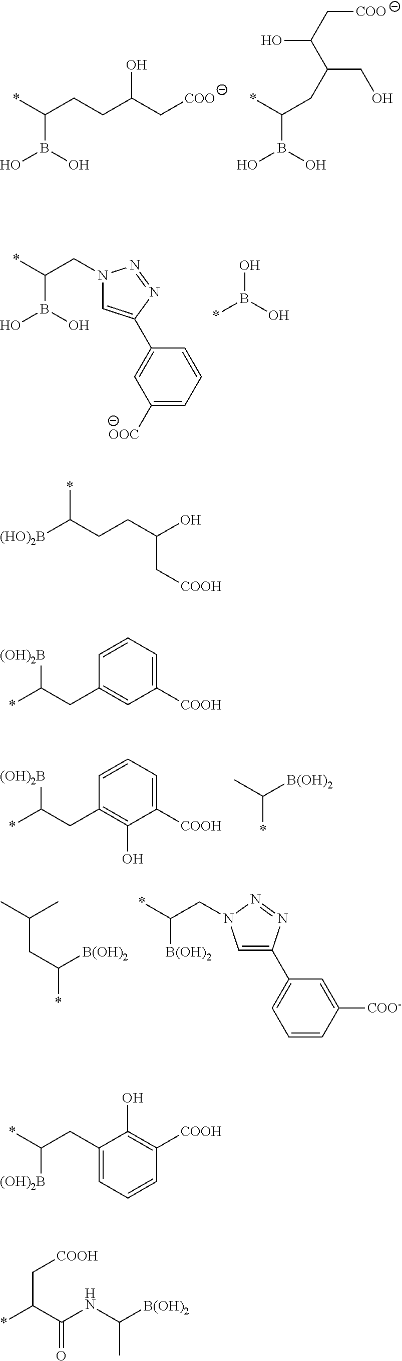

### Examples of Such Acyclic Boronic Acids Include:

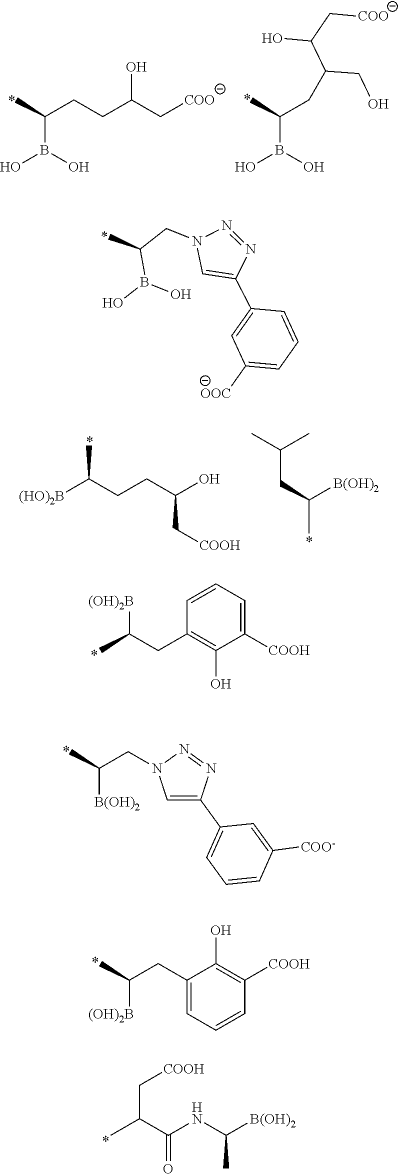

### Preferred Examples of Such Acyclic Boronic Acids Include:

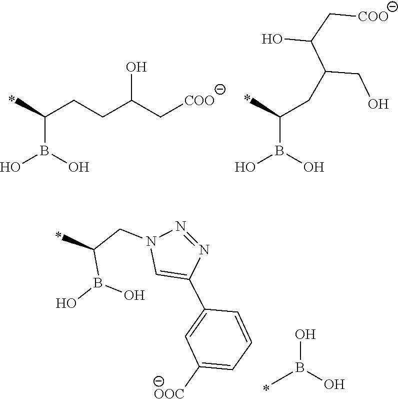

where * denotes the point of attachment of the hydrophilic group to the remainder of the molecule, e.g. to a linker group L as herein defined.
   Preferably, Group W is Selected from the Following Groups:

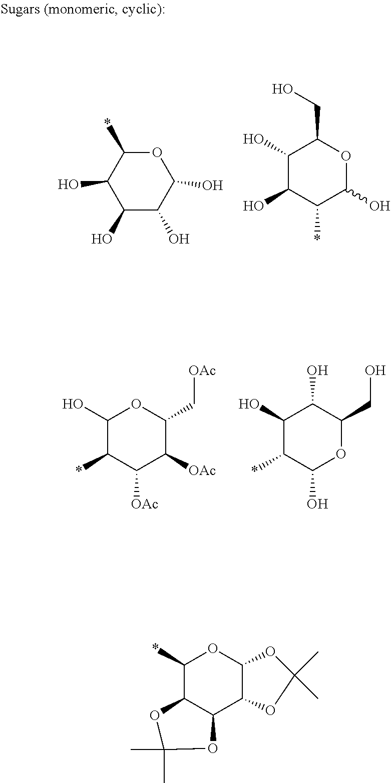

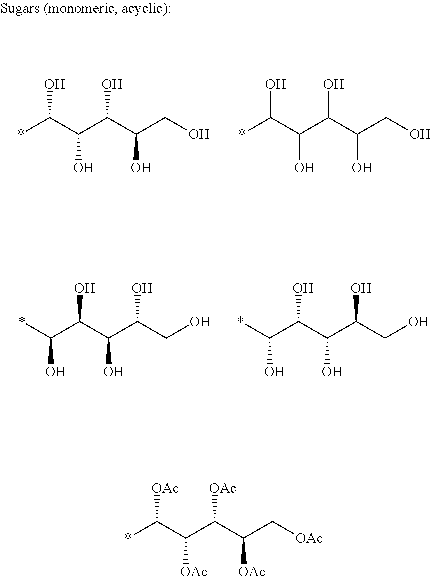

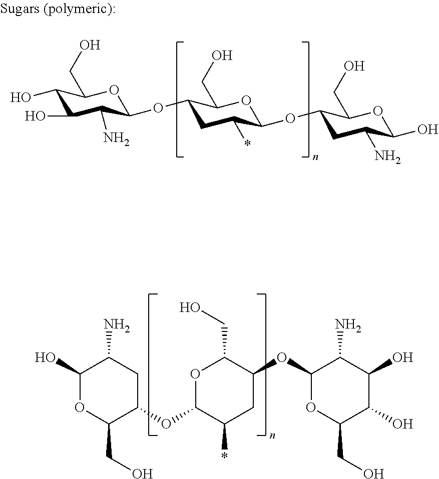

Any of the zinc chelators disclosed in WO 2018/033719, including any stereoisomers or pharmaceutically acceptable salts thereof, may be employed as the selective zinc-chelator compound in the present invention. The entire contents of WO 2018/033719 are incorporated herein by reference. For example, the selective zinc-chelator for use in the invention may be a compound according to any one of Examples 14, 22a, 22b, 23, 24, 25, 26, 33, 38, 41, 43, 44, 53, 54, 55, 56, 57, 58, 60, 67, 68a, 68b, 69, 70, 71, 74, 75, 77, 78, 79, 80, 81, 83a, 83b, 84, 85, 91, 96e, 97, 98, 99, 99d, 100, 101a, 101b, 103, 104, 106, 106b, 106c, 107, 120, 122, 123, 124a, 124b, 148, 149, 156b, 160, 163b, 182, 185, 187, 188, 189, 190, and 192 of WO 2018/033719, or a stereoisomer or pharmaceutically acceptable salt thereof.

Specific examples of zinc-chelators for use in the invention include, but are not limited to, the following:

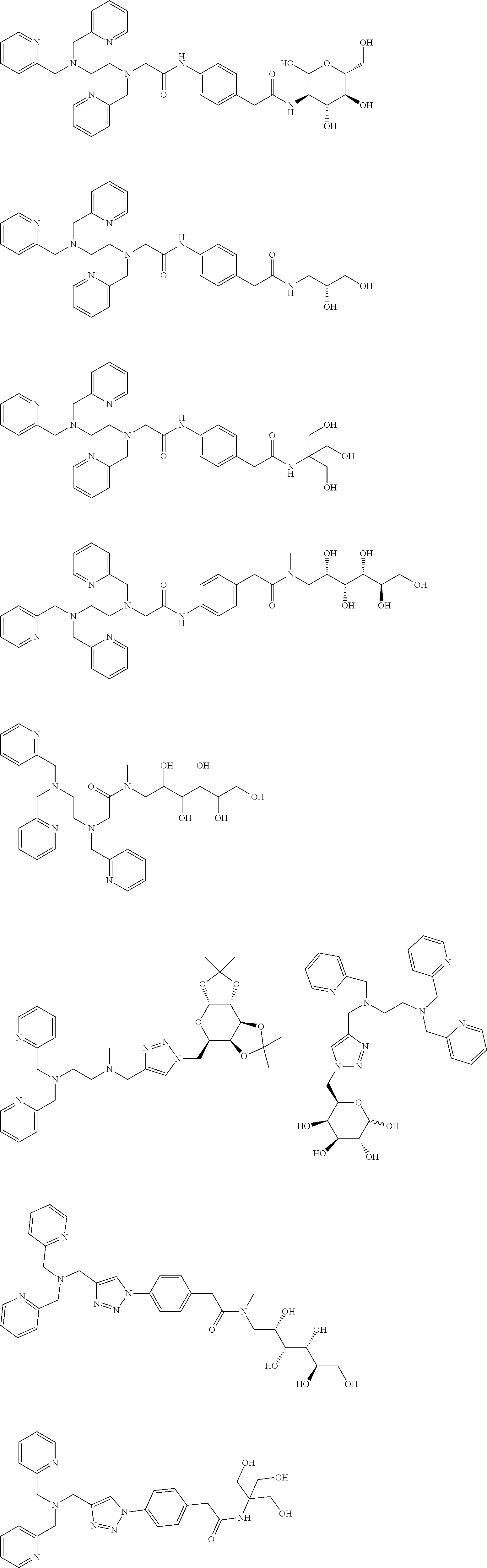

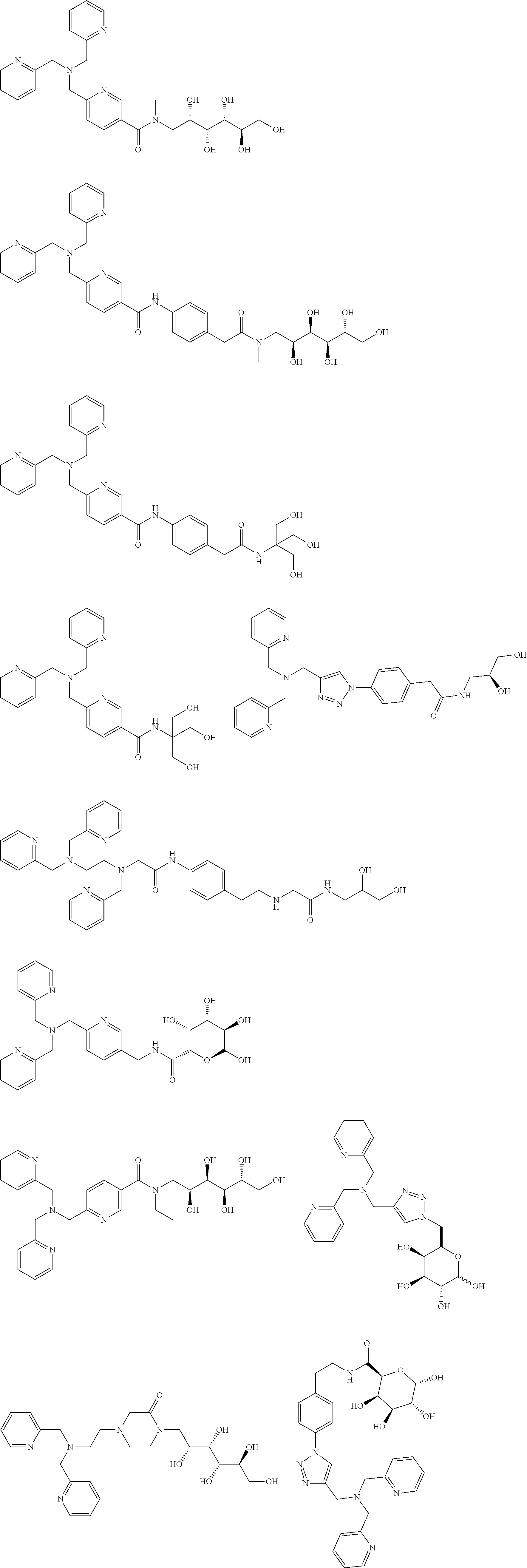

their stereoisomers, pharmaceutically acceptable salts and prodrugs thereof.

In some embodiments, the selective zinc-chelator for use in the invention is selected from the following compounds:

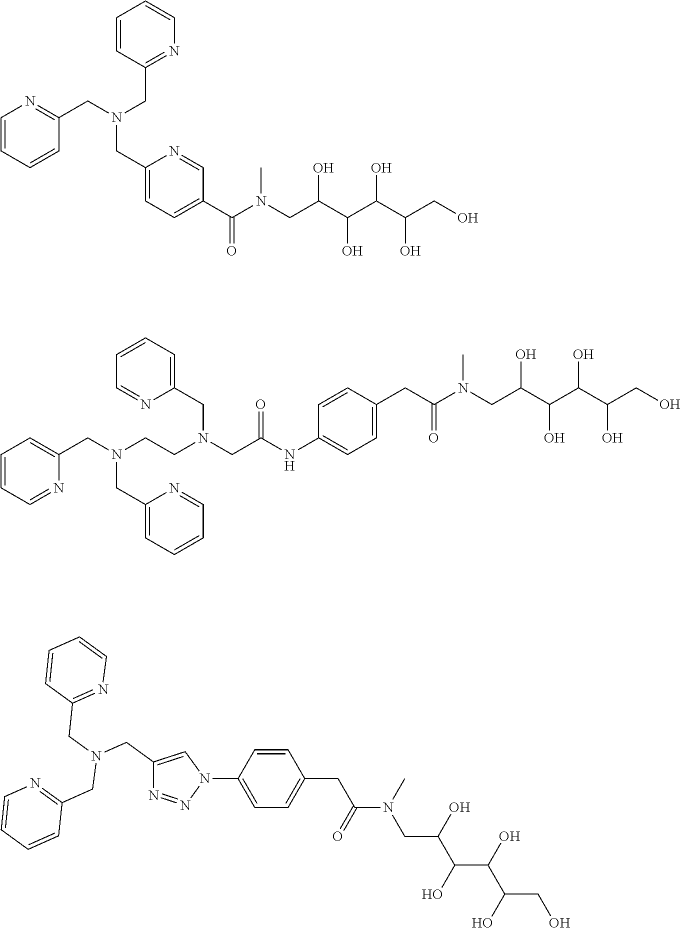 [SMILES: CN(CC(O)C(O)C(O)C(O)CO)C(=O)c1ccc(CN(Cc2ccccn2)Cc2ccccn2)nc1 ; CN(CC(O)C(O)C(O)C(O)CO)C(=O)Cc1ccc(NC(=O)CN(CCN(Cc2ccccn2)Cc2ccccn2)Cc2ccccn2)cc1 ; CN(CC(O)C(O)C(O)C(O)CO)C(=O)Cc1ccc(-n2cc(CN(Cc3ccccn3)Cc3ccccn3)nn2)cc1]

their stereoisomers, pharmaceutically acceptable salts and prodrugs thereof.

In some embodiments, the selective zinc-chelator for use in the invention is selected from the following compounds:

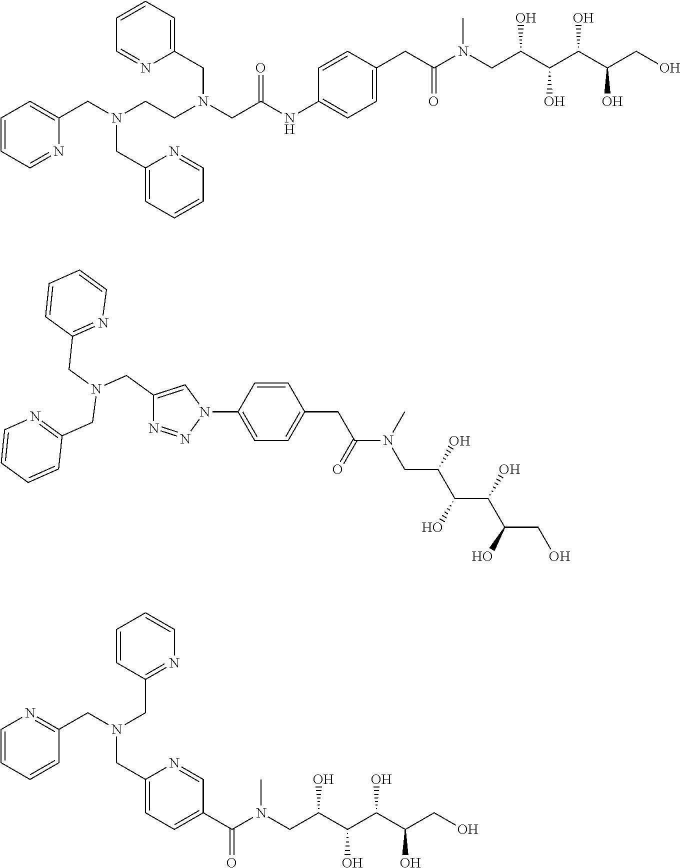 [SMILES: CN(C[C@H](O)[C@@H](O)[C@H](O)[C@H](O)CO)C(=O)Cc1ccc(NC(=O)CN(CCN(Cc2ccccn2)Cc2ccccn2)Cc2ccccn2)cc1 ; CN(C[C@H](O)[C@@H](O)[C@H](O)[C@H](O)CO)C(=O)Cc1ccc(-n2cc(CN(Cc3ccccn3)Cc3ccccn3)nn2)cc1 ; CN(C[C@H](O)[C@@H](O)[C@H](O)[C@H](O)CO)C(=O)c1ccc(CN(Cc2ccccn2)Cc2ccccn2)nc1]

their pharmaceutically acceptable salts, and prodrugs thereof.

In one embodiment, the selective zinc-chelator for use in the invention is the compound of Example 26 in WO 2018/033719 having the following structure, or a stereoisomer or pharmaceutically acceptable salt thereof:

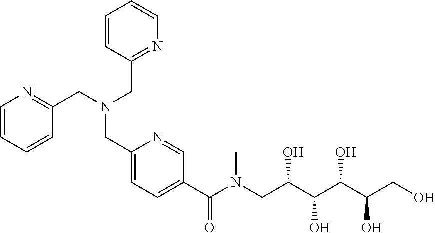 [SMILES: CN(C[C@H](O)[C@@H](O)[C@H](O)[C@H](O)CO)C(=O)c1ccc(CN(Cc2ccccn2)Cc2ccccn2)nc1]

In one embodiment, the selective zinc-chelator for use in the invention is an acetylated derivative of this compound, or a pharmaceutically acceptable salt thereof. For example, the selective zinc-chelator may be the compound of Example 189 of WO 2018/033719 having the following structure, or a stereoisomer or pharmaceutically acceptable salt thereof:

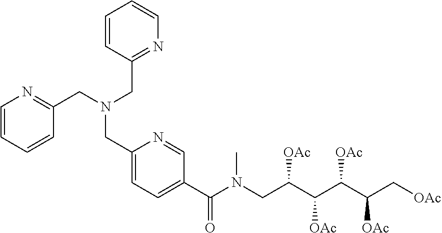 [SMILES: CC(=O)OC[C@@H](OC(C)=O)[C@@H](OC(C)=O)[C@H](OC(C)=O)[C@H](CN(C)C(=O)c1ccc(CN(Cc2ccccn2)Cc2ccccn2)nc1)OC(C)=O]

The second agent (B) for use in the invention is a β-lactam antibiotic selected from the group consisting of the penicillins, monobactams and carbapenems.

In some embodiments, the second agent (B) is a penicillin.

In some embodiments, the penicillin may be selected from the group consisting of cloxacillin, dicloxacillin, flucloxacillin, methicillin, nafcillin, oxacillin, ampicillin, amoxicillin, pivampicillin, bacampicillin, metampicillin, talampicillin, hetacillin, epicillin, phenoxymethylpenicillin, benzylpenicillin, carboxypenicillin, carbenicillin, ticarcillin, temocillin, mezlocillin, piperacillin and azlocillin. Any of these may be used in the form of a pharmaceutically acceptable salt.

In some embodiments, the penicillin is ampicillin, amoxicillin, temocillin, piperacillin, pivampicillin, or any pharmaceutically acceptable salt thereof. In some embodiments, the penicillin is ampicillin, amoxicillin, temocillin, piperacillin, or any pharmaceutically acceptable salt thereof. In some embodiments, the penicillin is amoxicillin or a pharmaceutically acceptable salt thereof.

In some embodiments, the penicillin is piperacillin, amoxicillin, ampicillin, or a pharmaceutically acceptable salt thereof.

In some embodiments, the second agent (B) is a monobactam. Monobactams are monocyclic, bacterially-produced β-lactam antibiotics or chemically synthesised equivalents thereof.

Any known monobactam antibiotic may be used in the invention. Non-limiting examples of monobactams include aztreonam, aztreonam lysine, tigemonam, nocardicin A, tabtoxin, BAL 30072, SYN 2416 (BAL 19764), carumonam, AIC 499, BOS 228 (LYS 228), MC-1, and their pharmaceutically acceptable salts. Some of these monobactams are shown in the following table:

| Compound | Name + Supplier |
|---|---|
| 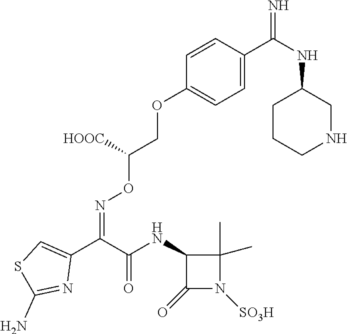 [SMILES: CC1(C)[C@H](NC(=O)/C(=N\O[C@@H](COc2ccc(C(=N)N[C@@H]3CCCNC3)cc2)C(=O)O)c2csc(N)n2)C(=O)N1S(=O)(=O)O] | AlC499 + BLI AiCuris |
| 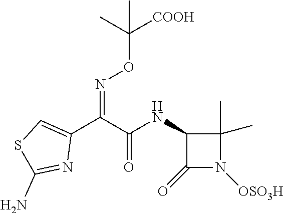 | Aztreonam Lysine Abbvie Gilead Sciences Pfizer/Astra Zeneca |
| 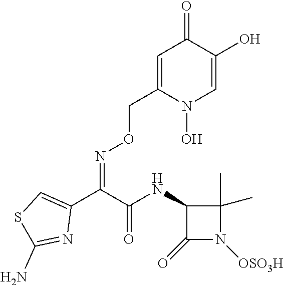 | BAL30072 |
| 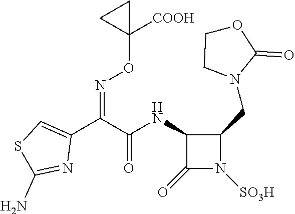 [SMILES: Nc1nc(/C(=N/OC2(C(=O)O)CC2)C(=O)N[C@@H]2C(=O)N(S(=O)(=O)O)[C@@H]2CN2CCOC2=O)cs1] | BOS228/LYS228 Boston Pharmaceuticals |
| 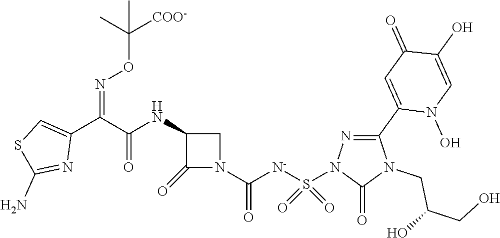 | MC-1 Pfizer |

In some embodiments, the monobactam for use in the invention is aztreonam or a pharmaceutically acceptable salt thereof. Aztreonam is a synthetic version of a chemical obtained from the bacterium Chromobacterium violaceum.

In some embodiments, the second agent (B) is a carbapenem. Carbapenems are β-lactam antibiotics which kill bacteria by binding to penicillin-binding proteins thus inhibiting bacterial cell wall synthesis.

Examples of carbapenems that may be used in the invention include, but are not limited to, benapenem, biapenem, doripenem, ertapenem, imipenem, lenapenem, meropenem, panipenem, razupenem, tebipenem, tebipenem (e.g. tebipenem pivoxil), thienpenem (also known as thienamycin), tomopenem and derivatives thereof. Derivatives include prodrug forms such as any of those herein described (e.g. esters), and any pharmaceutically acceptable salts thereof.

In some embodiments, the carbapenem is selected from meropenem, doripenem, imipenem, tebipenem, and any derivatives thereof. In some embodiments, the carbapenem is meropenem or a derivative thereof. In some embodiments, the carbapenem is meropenem. Preferred derivatives of carbapenems include the pharmaceutically acceptable salts thereof, for example the sodium salts. In one embodiment, the carbapenem is meropenem in the form of its sodium salt.

The third agent (C) for use in the invention is a serine β-lactamase inhibitor. A serine β-lactamase inhibitor is a compound which inhibits the activity of at least one serine β-lactamase (SBL) in the Ambler classes A, C or D. Serine β-lactamases (SBLs) are characterised by an active site serine.

In some embodiments, the third agent (C) is one which inhibits at least one SBL in Ambler class A, for example CepA, KPC-2, IMI-1, SME-1, PC1, TEM-1, TEM-2, TEM-3, TEM-30, TEM-50, SHV-1, SHV-2, SHV-10, CTX-M-15, PER-1, VEB-1, PSE-1, CARB-3, or RTG-4. In some embodiments, the third agent (C) is one which inhibits at least one SBL in Ambler class C, for example AmpC, CMY-1, ACT-1, FOX-1, MIR-1, GC1, CMY-10, CMY-19, or CMY-37. In some embodiments, the third agent (C) is one which inhibits at least one SBL in Ambler class D, for example OXA-1, OXA-10, OXA-11, OXA-15, OXA-23, OXA-48.

In some embodiments, the serine β-lactamase inhibitor for use in the invention is a diaza-bicyclo-octanone (DBO) compound, a prodrug or a pharmaceutically acceptable salt thereof. Such compounds are well known and documented in the art. Suitable examples include, but are not limited to, any of the compounds described in the following patent publications, the entire contents of which are incorporated herein by reference: WO 2020/030761, WO 2018/208557, WO 2021/041616, US 2015/0374673, U.S. Pat. No. 10,722,521, WO 02/10172, U.S. Pat. No. 9,695,122, WO 2016/157057, WO 2013/149121, WO 2018/053215, WO 2017/055922, WO 2019/145784, WO 2016/156348, WO 2016/177862, WO 2018/141986, WO 2018/060484, WO 2014/091268, WO 2016/116878, WO 2009/091856, WO 2013/038330 and WO 2013/030733.

Non-limiting examples of serine β-lactamase inhibitors for use in the invention include the following compounds, their pharmaceutically acceptable salts and prodrugs:

| Compound | Name + Supplier |
|---|---|
| 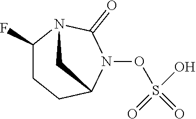 | ANT3310 Antabio |
| 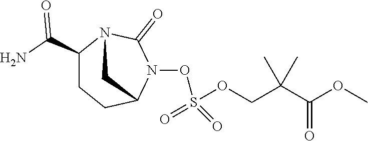 | ARX-1796 (AV006) Pfizer/ Arixa Pharmaceuticals |
| 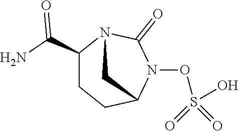 | Avibactam Abbvie/Pfizer Avycaz ® Zavicefta ® |
| 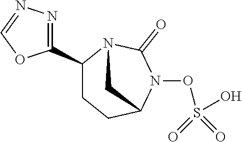 | CB-618 (MK-6183) |
| 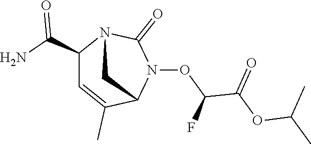 | ETX0282 Entasis Therapeutics |
| 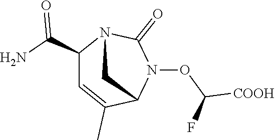 | ETX1317 Entasis Therapeutics |
| 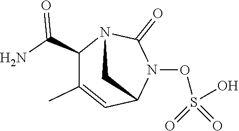 | Durlobactam ETX-2514 ® Entasis Therapeutics |
| 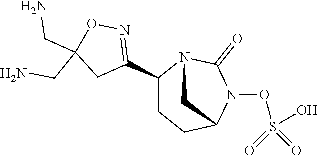 | GT-055 (LCB18-055) Geom Therapeutics Legochem B. |
| 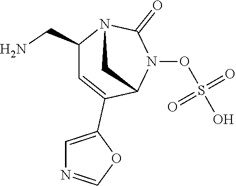 | Dabocin A Mutabilis |
| 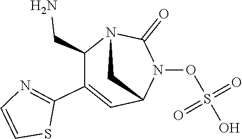 | Dabocin B Mutabilis |
| 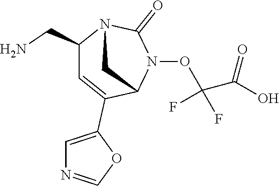 | Dabocin C Mutabilis |
| 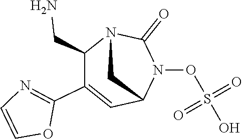 | Dabocin D Mutabilis |
| 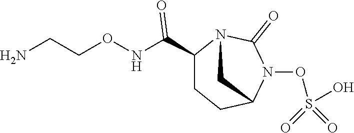 | Nacubactam OP0595 ® NacuGen Therapeutics/Roche |
| 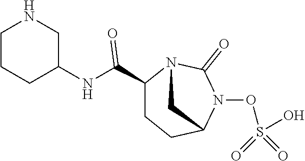 | Relebactam Recarbrio ® Merck & Co. |
| 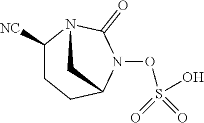 | WCK4234 |
| 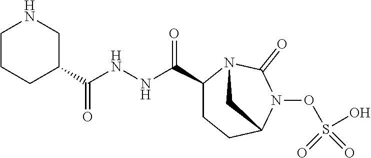 | Zidebactam WCK 5222 ® WCK 6777 ® Wockhardt Ltd. |

In one embodiment, the serine β-lactamase inhibitor for use in the invention is avibactam or a pharmaceutically acceptable salt thereof. In one embodiment, avibactam is provided in the form of the sodium salt.

Salts of avibactam for use in the invention may be provided in any known polymorphic or pseudopolymorphic forms (i.e. “crystalline forms”) such as, but not limited to, the crystalline forms disclosed in WO 2011/042560, the entire content of which is incorporated herein by reference. In one embodiment, a salt of avibactam may be used in crystalline Form “A”, “B”, “C”, “D” or “E”, preferably in crystalline “Form B”. In one embodiment, the sodium salt of avibactam may be used in crystalline form “A”, “B”, “C”, “D” or “E”, preferably in crystalline “Form B”. Any crystalline form may be provided in anhydrous or in hydrated form.

In some embodiments, the serine β-lactamase inhibitor for use in the invention is a non-antibiotic β-lactam. As used herein, the term “non-antibiotic β-lactam” is intended to refer to a β-lactam compound having negligible intrinsic antimicrobial activity. Non-limiting examples of such compounds include clavulanic acid, sulbactam, tazobactam and enmetazobactam, their pharmaceutically acceptable salts or prodrugs thereof. In some embodiments, the serine β-lactamase inhibitor is sulbactam, tazobactam or a pharmaceutically acceptable salt or prodrug thereof.

In some embodiments, the serine β-lactamase inhibitor for use in the invention is avibactam, sulbactam, tazobactam, or a pharmaceutically acceptable salt or prodrug thereof.

In preferred embodiments of any of the methods, uses, combinations or pharmaceutical compositions herein described, the selective zinc-chelator (A) is the following compound, a stereoisomer, a pharmaceutically acceptable salt, or a prodrug thereof:

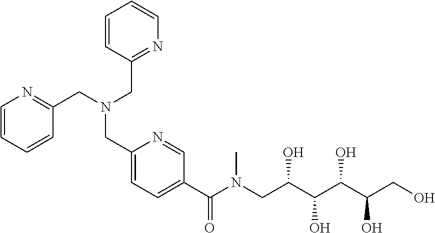 [SMILES: CN(C[C@H](O)[C@@H](O)[C@H](O)[C@H](O)CO)C(=O)c1ccc(CN(Cc2ccccn2)Cc2ccccn2)nc1]

and either:
the second agent (B) is meropenem or a pharmaceutically acceptable salt thereof, and the third agent (C) is avibactam or a pharmaceutically acceptable salt thereof;
or
the second agent (B) is meropenem or a pharmaceutically acceptable salt thereof, and the third agent (C) is sulbactam or a pharmaceutically acceptable salt thereof;
or
the second agent (B) is meropenem or a pharmaceutically acceptable salt thereof, and the third agent (C) is tazobactam or a pharmaceutically acceptable salt thereof;
or
the second agent (B) is piperacillin or a pharmaceutically acceptable salt thereof, and the third agent (C) is tazobactam or a pharmaceutically acceptable salt thereof;
or
the second agent (B) is ampicillin or a pharmaceutically acceptable salt thereof, and the third agent (C) is sulbactam or a pharmaceutically acceptable salt thereof; or
the second agent (B) is amoxicillin or a pharmaceutically acceptable salt thereof, and the third agent (C) is sulbactam or a pharmaceutically acceptable salt thereof;
or
the second agent (B) is aztreonam or a pharmaceutically acceptable salt thereof, and the third agent (C) is avibactam or a pharmaceutically acceptable salt thereof;
or
the second agent (B) is piperacillin or a pharmaceutically acceptable salt thereof, and the third agent (C) is avibactam or a pharmaceutically acceptable salt thereof.

In preferred embodiments of any of the methods, uses, combinations or pharmaceutical compositions herein described, the selective zinc-chelator (A) is the following compound, a stereoisomer, a pharmaceutically acceptable salt, or a prodrug thereof:

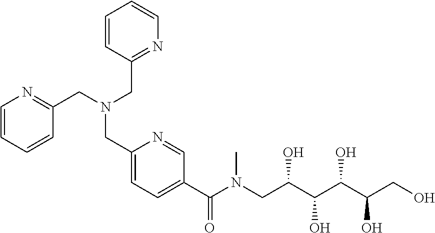 [SMILES: CN(C[C@H](O)[C@@H](O)[C@H](O)[C@H](O)CO)C(=O)c1ccc(CN(Cc2ccccn2)Cc2ccccn2)nc1]

and either:
the second agent (B) is meropenem or a pharmaceutically acceptable salt thereof, and the third agent (C) is avibactam or a pharmaceutically acceptable salt thereof;
or
the second agent (B) is aztreonam or a pharmaceutically acceptable salt thereof, and the third agent (C) is avibactam or a pharmaceutically acceptable salt thereof.

Any of the compounds (A), (B) and (C) herein described may be provided in the form of a pharmaceutically acceptable salt. The compounds may be converted into a salt thereof, particularly into a pharmaceutically acceptable salt thereof with an inorganic or organic acid or base. Acids which may be used for this purpose include hydrochloric acid, hydrobromic acid, sulphuric acid, sulphonic acid, methanesulphonic acid, phosphoric acid, fumaric acid, succinic acid, lactic acid, citric acid, tartaric acid, maleic acid, acetic acid, trifluoroacetic acid and ascorbic acid. Bases which may be suitable for this purpose include alkali and alkaline earth metal hydroxides, for example sodium hydroxide, potassium hydroxide or cesium hydroxide, ammonia and organic amines such as diethylamine, triethylamine, ethanolamine, diethanolamine, cyclohexylamine and dicyclohexylamine. Procedures for salt formation are conventional in the art.

In one embodiment, the compounds may be formulated in the form of calcium or zinc salts. This may be advantageous in contributing to a balance of these ions in the body. Sources of calcium or zinc which can be used for this purpose include, but are not limited to, calcium or zinc chloride, calcium or zinc carbonate, calcium or zinc gluconate, and calcium or zinc-edetate. Preferred are calcium or zinc salts in clinical use and which are on the FDA GRAS list, for example any commercially available calcium gluconate or zinc gluconate salts of any of the compounds herein described.

A challenge related to many conventional antibiotics is their low oral bioavailability, i.e. a low percentage uptake from the gastrointestinal system to the blood following oral administration. The cause of this challenge is often related to a low log P or high water-solubility of the compound due to an excess of polar functional groups which form hydrogen bonds with the surrounding biological fluid. To address this challenge, hydrolytically or enzymatically cleavable derivatives of such polar functional groups may be used to eliminate the ability for hydrogen bonding and thus improve oral bioavailability. Such modified derivatives of any of the compounds herein described are examples of “prodrugs” that may be used in the invention.

Any of the compounds (A), (B) and (C) herein described may be provided in the form of a “prodrug”. The term “prodrug” refers to a derivative of an active compound which undergoes a transformation under the conditions of use, for example within the body, to release an active drug. A prodrug may, but need not necessarily, be pharmacologically inactive until converted into the active drug. As used herein, the term “prodrug” extends to any compound which under physiological conditions is converted into any of the agents herein described.

Suitable prodrugs include compounds which are hydrolysed under physiological conditions to the desired molecule. Prodrugs may typically be obtained by masking one or more functional groups in the parent molecule which are considered to be, at least in part, required for activity using a pro-group. By “pro-group” as used herein is meant a group which is used to mask a functional group within an active drug and which undergoes a transformation, such as cleavage, under the specified conditions of use (e.g. administration to the body) to release a functional group and hence provide the active drug. Pro-groups are typically linked to the functional group of the active drug via a bond or bonds that are cleavable under the conditions of use, e.g. in vivo. Cleavage of the pro-group may occur spontaneously under the conditions of use, for example by way of hydrolysis, or it may be catalysed or induced by other physical or chemical means, e.g. by an enzyme, or by exposure to a change in pH, etc. Where cleavage is induced by other physical or chemical means, these may be endogenous to the conditions of use, for example pH conditions at a target site, or these may be supplied exogenously.

A wide variety of pro-groups suitable for masking functional groups in active compounds to provide prodrugs are well known in the art. For example, a hydroxy functional group may be masked as an ester, e.g. acetate esters, a phosphate ester, or a sulfonate ester which may be hydrolysed in vivo to provide the parent hydroxy group. An amide functional group may be hydrolysed in vivo to provide the parent amino group. A carboxyl group may be masked as an ester or amide which may be hydrolysed in vivo to provide the parent carboxyl group. Other examples of suitable pro-groups will be apparent to those of skill in the art.

In one embodiment, the compounds for use in the invention have a hydroxy functional group that can be derivatised to produce suitable prodrugs. For example, the hydroxy group in a parent molecule can be converted to an alkyl or aryl ester, a phosphate ester, or a sulfonate ester. Non-limited examples of such derivatisation are illustrated below:

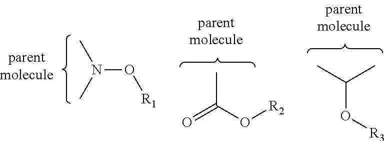

In the parent molecule of the prodrug, R₁, R₂ and R₃═H. In the corresponding prodrug, R₁, R₂ and R₃ may be a group exemplified by the following groups:

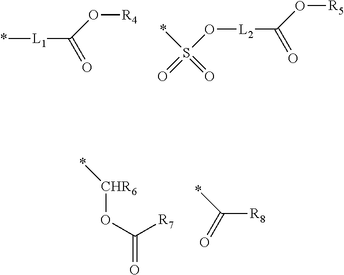

wherein R₄ to R₈ are independently selected from H, C₁₋₆ haloalkyl and C₁₋₆ alkyl;
and L₁ and L₂ are linking groups, for example an optionally halogenated, straight chained or branched, C₁₋₆ alkylene or C₁₋₆ alkenylene group.

The combination of agents (A), (B) and (C) herein described finds use in therapy. The invention thus provides compositions and combinations of these agents as described herein for use as a medicament or for use in therapy.

The agents, compositions and combinations herein described may be used in the treatment of a bacterial infection in a subject. The present invention therefore provides methods of treatment of bacterial infections in a subject in need thereof, which methods comprise administering to said subject an effective amount of such agents, compositions or combinations as described herein. The invention also provides agents, compositions or combinations as herein described for use in treating a bacterial infection. It also provides the use of agents, compositions or combinations as herein described in the manufacture of a medicament for use in the treatment of a bacterial infection.

In some embodiments, the subject is a human or non-human mammal. The term “mammal” is used in its usual biological context and includes humans, dogs, cats, horses, cattle, dogs, cats, rats and mice. In further embodiments, the subject is a human.

The term “bacterial infection” refers to the invasion of the host organism by one or more species of pathogenic bacteria. A bacterial infection will generally be understood to occur when the presence of the bacterial population causes damage or harm to the host organism, i.e. to the subject, for example damage or harm to the host's cells, tissues or organs.

As used herein, the terms “treat, “treatment” or “treating” refer to administration of an agent or combination of agents for therapeutic or prophylactic purposes. Therapeutic treatment is effective to relieve, to some extent, one or more symptoms of the infection. It refers to administering treatment to a subject that is already suffering from an infection. In some embodiments, therapeutic treatment includes clinical cure, i.e. elimination of symptoms of an active infection, including elimination of the underlying cause of the infection (i.e. viable microbes involved in the infection). Prophylactic treatment refers to the treatment of a subject who is not yet infected, but who is susceptible to, or otherwise at risk of, an infection. Prophylactic treatment reduces the chances that the subject will develop an infection.

Bacterial infections that can be treated in accordance with the invention can comprise a wide spectrum of bacteria. Example organisms include gram-positive and gram-negative bacteria.

In some embodiments, the bacterial infection is associated with gram-positive and/or gram-negative bacteria. In some embodiments, the bacterial infection is associated with gram-negative bacteria.

In some embodiments, the infection is associated with gram-positive or gram-negative bacteria which are resistant to treatment with one or more conventional antibiotics when administered alone, particularly bacteria that are resistant to treatment with β-lactam antibiotics.

In some embodiments, the bacterial infection is associated with gram-positive or gram-negative bacteria which produce metallo-β-lactamases. In some embodiments, the bacterial infection is associated with gram-negative bacteria which produce metallo-β-lactamases. In some embodiments, the infection is associated with gram-negative multi-resistant bacteria harbouring extended spectrum metallo-β-lactamases (ESBL).

In some embodiments, the infection is associated with gram-positive or gram-negative bacteria which produce serine-β-lactamases. In some embodiments, the bacterial infection is associated with gram-negative bacteria which produce serine-β-lactamases.

The agents, compositions and combinations herein described find particular use in the treatment of bacterial infections caused by bacteria which are resistant to treatment with antibiotics when administered alone, particularly where the resistance is caused by SBLs. The agents, compositions and combinations are therefore useful in the elimination or reduction of antibiotic resistance, in particular in gram-negative bacteria. In particular, they are useful in eliminating or reducing resistance caused by SBLs.

In some embodiments, the agents, compositions and combinations herein described may be used in the treatment of a bacterial infection which occurs after a relapse following an antibiotic treatment. These can therefore be used in the treatment of a subject (e.g. a patient) who has previously received antibiotic treatment for the bacterial infection.

In some embodiments, the infection is caused by a bacteria selected from the group consisting of Acinetobacter baumannii, Acinetobacter calcoaceticus, Acinetobacter haemolyticus, Aeromonas spp, Aeromones hydrophilia, Bacillus cereus Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Bacteroides thetaiotaomicron, Borrelia burgdorferi, Bordetella pertussis, Bordetella parapertussis, Bordetella bronchiseptica, Burkholderia cepacia, Branhamella catarrhalis, Campylobacterfetus, Campylobacter jejuni, Campylobacter coli, Chryseobacterium indologenes Citrobacter freundii, Clostridium difficile, Corynebacterium diphtheriae, Corynebacterium ulcerans, Elizabethkingia meningoseptica, Escherichia coli, Enterobacter cloacae, Enterobacter aerogenes, Enterococcus faecalis, Enterococcus faecium, Francisella tularensis, Gardnerella vaginalis, Haemophilus influenzae, Haemophilus parainfluenzae, Haemophilus haemolyticus, Haemophilus parahaemolyticus, Haemophilus ducreyi, Helicobacter pylori, Legionella pneumophila, Listeria monocytogenes, Kingella, Moraxella, Klebsiella pneumoniae, Klebsiella oxytoca, Legionella pneumophila, Listeria monocytogenes, Morganella morganii, Mycobacterium tuberculosis, Mycobacterium avium, Mycobacterium intracellulare, Moraxella, Mycobacterium leprae, Myroides odoratimimus, Neisseria gonorrhoeae, Neisseria meningitidis Pasteurella multocida, Pasteurella haemolytica, Proteus mirabilis, Proteus vulgaris, Providencia alcalifaciens, Providencia rettgeri, Providencia stuartii, Pseudomonas fluorescens, Pseudomonas aeruginosa, Pseudomonasfluorescens, Pseudomonas acidovorans, Pseudomonas alcaligenes, Pseudomonasputida, Serratia marcescens, Salmonella typhimurium, Salmonella typhi, Salmonella paratyphi, Salmonella enteritidis, Serratia marcescens, Shigella dysenteriae, Shigella flexneri, Shigella sonnei, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, Staphylococcus saccharolyticus. Stenotrophomonas maltophilia, Streptococcus pneumoniae, Streptococcus agalactiae, Streptococcus pyogenes, Vibrio cholerae, Vibrio parahaemolyticus, Yersinia pestis, Yersinia pseudotuberculosis, Yersinia intermedia, Yersinia enterocolitica, and Yersinia pestis.

In some embodiments of the invention, the bacterial infection is caused by a bacteria selected from the group consisting of Klebsiella pneumoniae, Acinetobacter baumanii, Pseudomonas aeruginosa and Escherichia coli.

In some embodiments of the invention, the infection is caused by a bacteria selected from the group consisting of Klebsiella pneumoniae K66-45, Klebsiella pneumoniae ST147, Klebsiella pneumoniae ST101, Klebsiella pneumoniae BAA1705, Acinetobacter baumannii ST 25, Acinetobacter baumannii ST15, Pseudomonas aeruginosa ST773, and Pseudomonas aeruginosa ST111.

In some embodiments of the invention, the bacterial infection is caused by a bacteria selected from the group consisting of Klebsiella pneumoniae Kpn St147, Klebsiella pneumoniae ST101, Klebsiella pneumoniae K66-45 and Klebsiella pneumoniae BAA 1705.

In some embodiments, the agents, compositions and combinations herein described may be employed to treat a bacterial biofilm. As used herein, the term “bacterial biofilm” means a community of bacteria which are contained within an extracellular polymeric substance (EPS) matrix produced by the bacteria and attached to a body surface. Treatment of a bacterial biofilm may be effective to disrupt, remove or detach at least part of a bacterial biofilm. In some embodiments, treatment will be effective to eradicate a biofilm.

Preferred combinations of agents for use in the invention are those which demonstrate enhanced (e.g. synergistic) activity in any of the treatment methods herein described, for example in the treatment of a bacterial infection in a subject relative to the use of any one of the agents alone, preferably relative to the use of any two of the agents in combination. For example, the combinations of agents may demonstrate enhanced (e.g. synergistic) activity against one or more bacteria, such as Klebsiella pneumoniae Kpn St147, Klebsiella pneumoniae ST101, Klebsiella pneumoniae K66-45 and Klebsiella pneumoniae BAA1705 (or any of the other bacterial strains against which the combinations of agents are tested in the examples), relative to the use of any one of the agents alone, preferably relative to the use of any two of the agents in combination. Evidence of synergy may include any one of the following: a faster cure rate, cure time or symptom improvement (e.g. improvement in at least one sign or key symptom of bacterial infection); and a reduction in the relapse rate of bacterial infection (i.e. the rate of reappearance of the infection after cessation of the treatment).

In the context of the susceptibility of a microorganism to an antimicrobial agent, synergy may be understood with reference to the definition set out by The European Committee for Antimicrobial Susceptibility Testing (EUCAST) of the European Society of Clinical Microbiology and Infectious Diseases (ESCMID) 2000 (see EUCAST Definitive Document E. Def 1.2: Terminology relating to methods for the determination of susceptibility of bacteria to antimicrobial agents. Clin. Microbiol. Infect. 2000; 6(9): 503-508): FICI≤0.5; synergy.

In the combination therapy herein described, the agents (A), (B) and (C) are co-administered to the subject (e.g. a human patient). The term “co-administration” is used herein to refer to the delivery of two or more separate chemical entities in vivo. Co-administration includes the simultaneous delivery of separate agents; the simultaneous delivery of a combination of agents; as well as the delivery of one or two agents in combination followed by delivery of an additional agent or agents. In all cases, agents that are co-administered are intended to work in conjunction with each other, i.e. these will be present together in vivo, regardless of when or how they are actually administered, to achieve the desired therapeutic and/or prophylactic effect.

The agents for use in the invention may thus be administered simultaneously, separately or sequentially. Administration of each agent (whether separately or simultaneously) may be via any conventional administration route or routes, such as oral or parenteral administration.

The agents for use in the invention may be formulated together or they may be formulated separately. Where these are formulated separately, the agents (A), (B) and (C) may each be provided in a separate formulation. Alternatively, two of the agents may be provided in a single formulation and the third agent may be provided separately. In other words, the first agent (A) may be formulated with the second agent (B) and the third agent (C) may be formulated separately; or the first agent (A) may be formulated with the third agent (C) and the second agent (B) may be formulated separately; or the second agent (B) may be formulated with the third agent (C) and the first agent (A) may be formulated separately.

In some embodiments, the agents (A), (B) and (C) are administered simultaneously.

In one such embodiment, administration may be achieved by combining the agents into a single dosage form, i.e. into the same pharmaceutical composition. Simultaneous administration may also be achieved by administering the agents in different pharmaceutical compositions. These may be administered via the same route, such as orally or parenterally (e.g. intravenously). In another embodiment the agents are administered via different routes, for example one or two of the agents may be administered orally and one or two of the agents may be administered intravenously.

In some embodiments, the agents are administered sequentially. In one embodiment, the agents are administered via the same route, such as orally or parenterally (e.g. intravenously). In another embodiment the agents are administered via different routes, for example one or two of the agents may be administered orally and one or two of the agents may be administered intravenously.

Pharmaceutical compositions for use in the invention comprise one or more of the agents as herein defined and one or more pharmaceutically acceptable carriers, diluents or excipients. By “pharmaceutically acceptable” is meant that the ingredients must be compatible with other ingredients of the composition as well as physiologically acceptable to the recipient. Pharmaceutical compositions may be formulated according to techniques and procedures well known in the art and widely described in the literature and may comprise any of the known carriers, diluents or excipients. Other ingredients may also be included, according to techniques well known in the art, for example stabilisers, preservatives, etc. The compositions may be in the form of sterile aqueous solutions and/or suspensions of the pharmaceutically active ingredients, aerosols, ointments and the like. The compositions may also be in a sustained release form, for example microparticles, nanoparticles, emulsions, nano-suspensions, lipid particles or oils. The agents for use in the invention may also be provided in formulations of ZnO nanoparticles as described, for example, by Pati et al. in Nanomedicine (2014), 10(6), 1195-1208.

These may additionally be provided in the form of films, patches or folios having the selective zinc-chelator coated on the surface.

The compositions may be formulated according to techniques and procedures well known in the literature and may comprise any of known carriers, diluents or excipients. For example, compositions for use in the invention which are suitable for parenteral administration conveniently may comprise sterile aqueous solutions and/or suspensions of the active agent(s) preferably made isotonic with the blood of the recipient generally using sodium chloride, glycerin, glucose, mannitol, sorbitol and the like. In addition, the composition may contain any of a number of adjuvants, such as buffers, preservatives, dispersing agents, agents that promote rapid onset of action or prolonged duration of action.

Compositions suitable for oral administration may be in sterile purified stock powder form, preferably covered by an envelope or envelopes which may contain any of a number or adjuvants such as buffers, preservative agents, agents that promote prolonged or rapid release. Compositions suitable for local or topical administration may comprise the agent(s) mixed with known ingredients such as paraffin, vaseline, cetanol, glycerol and the like, to form suitable ointments or creams.

The active agent(s) in any pharmaceutical composition may comprise from 0.05% to 99% by weight of the composition. An appropriate amount of the agent(s) may readily be determined by the skilled person.

In some embodiments, agents (A), (B) and (C) may be formulated in the same pharmaceutical composition. Such compositions form a further aspect of the invention. The invention thus provides a pharmaceutical composition comprising an effective amount of the first agent (A), second agent (B) and third agent (C) herein described, together with at least one pharmaceutically acceptable diluent or carrier.

In some embodiments, each agent is present in the composition in a therapeutically effective amount. In some embodiments, each agent is present in a prophylactically effective amount. Such an amount may readily be determined by the skilled person, for example a physician (where the subject is a human patient).

In some embodiments, provided herein are pharmaceutical compositions in which the agents are present in synergistically effective amounts.

Any of the compositions described herein may be provided in unit dose form. By “unit dose form” is meant a composition containing one or more agents that is suitable for administration to a subject, preferably a human or non-human mammal, in a single dose. The preparation of a single or unit dose does not imply that it is administered once per day or once per course of therapy. It may be administered more than once per day (e.g. twice, thrice or more per day) and may be given more than once during the course of a therapy.

Administration of the agents herein described to a subject may be by any suitable method known in the medicinal arts, including intravenous, intracerebral, oral, parenteral, topical, subcutaneous, or inhalation. In the case of an antibacterial adjuvant, a suitable administration regime is continuous systemic administration of the adjuvant in combination with co-administration of the respective antibacterial agent(s). In the case of sepsis caused by resistant gram-negative bacteria, for example, the adjuvant may be intravenously continuously infused, while an antibacterial agent is administered as approved by the respective regulatory authorities.

In one embodiment, continuous intravenous infusion of any of the agents herein described may also be carried out simultaneously with administration of the other active agents via other administration routes, for example by inhalation, or by oral or topical administration. For inhalation, a micronized formulation of the agent is suitable. For oral administration, a tableted form of the agent is suitable.

It will be understood that any pharmaceutical composition containing an agent or combination of agents as herein described will be administered to the subject in an “effective amount”, i.e. in an amount that will elicit the biological or medical response of the patient that is being sought by the physician in the treatment as herein described, for example in an amount that will inhibit or eliminate bacterial growth. The “effective amount” may depend on factors such as the nature of the particular active agents, the choice of other non-active components, the severity of the condition, the timing and duration of the treatment, whether the treatment is intended to be therapeutic or prophylactic, etc. In one embodiment, the effective amount is a “therapeutically effective amount” for the alleviation, to some extent, of one or more symptoms of the infection or for clinical cure, i.e. elimination of symptoms of an active infection, including elimination of the underlying cause of the infection (i.e. viable microbes involved in the infection). In one embodiment, the effective amount is a “prophylactically effective amount” for prophylaxis of the symptoms of the condition being prevented.

In the case of an antibacterial agent or adjuvant to an antibacterial agent, this may be administered in a single dose to be taken at regular intervals e.g. once or twice a day, once every 48 hours or once every 72 hours. Sustained formulations may be given at longer intervals, e.g. 1 to 2 times a month or every three months.

Appropriate dosages of each agent to be administered will depend on the nature of the agent, the precise condition to be treated and its severity, the age and weight of the subject to which it is administered (e.g. a patient), the manner and schedule of administration (e.g. the number of daily or monthly doses and the length of the course of treatment) etc. and may be routinely determined by the skilled practitioner.

In some embodiments, the first agent (A) is administered in a dosage range from about 1 to 200 mg/kg body weight, for example from 1 to 100 mg/kg body weight, e.g. 5 to 70 mg/kg body weight, 5 to 50 mg/kg body weight, 10 to 70 mg/kg body weight, or 10 to 50 mg/kg body weight. Administration of the agent (A) can be via any of the accepted modes of administration including, but not limited to, intravenous, intracerebral, oral, parenteral, topical, subcutaneous, or inhalation. Oral or intravenous administration may be preferred. More preferably, the agent (A) may be administered intravenously.

In some embodiments, the second agent (B) is administered in a dosage range from about 1 to 200 mg/kg body weight, for example from 1 to 100 mg/kg body weight, e.g. 5 to 70 mg/kg body weight, 5 to 50 mg/kg body weight, 10 to 70 mg/kg body weight, or 10 to 50 mg/kg body weight. Administration of the agent (B) can be via any of the accepted modes of administration including, but not limited to, intravenous, intracerebral, oral, parenteral, topical, subcutaneous, or inhalation.

Oral or intravenous administration may be preferred. More preferably, the agent (B) may be administered intravenously. Where the second agent is meropenem and the subject is a human patient, it may be administered intravenously at the recommended dosage of about 1 g per 70 kg bodyweight, for example.

In some embodiments, the third agent (C) is administered in a dosage range from about 1 to 200 mg/kg body weight, for example from 1 to 100 mg/kg body weight, e.g. 1 to 70 mg/kg body weight, 1 to 50 mg/kg body weight, 5 to 30 mg/kg body weight, or 5 to 20 mg/kg body weight. Administration of the agent (C) can be via any of the accepted modes of administration including, but not limited to, intravenous, intracerebral, oral, parenteral, topical, subcutaneous, or inhalation. Oral or intravenous administration may be preferred. More preferably, the agent (C) may be administered intravenously. Where the third agent is avibactam and the subject is a human patient, it may be administered intravenously at the recommended dosage of about 0.5 g per 70 kg bodyweight, for example.

Some embodiments of the invention include a kit comprising the agents (A), (B) and (C) as herein described. In such a kit, each agent may be provided in a separate container. In other embodiments, two of the agents may be provided in the same container and the third may be provided in a separate container. Each container may include a solid, solution or dispersion. Where any of the agents are solids intended for reconstitution, the kit may additionally contain a diluent suitable for preparation of the intended formulation for administration. Optionally, a kit may additionally contain instructions relating to the use of the agents in a method of treatment as herein described.

As herein described, the invention relates to a combination therapy which involves the co-administration of three agents, i.e. (A), (B) and (C). In some embodiments, the therapy may further comprise the step of administration to the subject of one or more additional antibacterial and/or adjuvant agents generally known and used in the art. However, in some embodiments, the therapy is a “triple combination” therapy in which only the three agents (A), (B) and (C) as herein described are employed as active agents, i.e. no additional antibacterial or adjuvant agents are required to treat and/or prevent the bacterial infection or the bacterial biofilm. In one set of embodiments, the methods of medical treatment according to the invention thus consist essentially of (e.g. consist of) the steps herein described. In another set of embodiments, the medical uses according to the invention consist essentially of (e.g. consist of) co-administration of (A), (B) and (C) as herein described. In another set of embodiments, the pharmaceutical compositions according to the invention consist essentially of (e.g. consist of) the agents (A), (B) and (C).

The invention is described in more detail in the following examples. These are used for the purposes of illustration only and should not be considered limiting.

### EXAMPLES

### Example 1—Minimal Inhibitory Concentration (MIC) Determination of Meropenem (MEM) in Combination with a Zinc Chelator and Avibactam Vs. Klebsiella pneumoniae Kpn St147, Klebsiella pneumoniae ST101, Klebsiella pneumoniae K66-45 and Klebsiella pneumoniae BAA1705 (Checkerboard Assay)

The following compound was employed as the zinc chelator and synthesised according to Example 26 of WO 2018/033719:

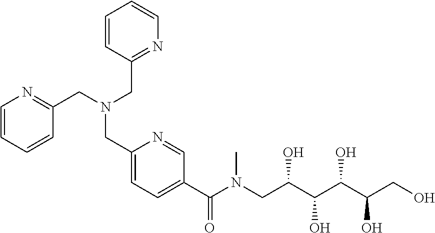 [SMILES: CN(C[C@H](O)[C@@H](O)[C@H](O)[C@H](O)CO)C(=O)c1ccc(CN(Cc2ccccn2)Cc2ccccn2)nc1]

Avibactam was purchased from Biosynth (https://www.biosynth.com/) and meropenem (MEM) was purchased from AdooQ Bioscience (https://www.adooq.com/meropenem.html).

Antimicrobial susceptibility testing (AST) by broth microdilution was performed according to The European Committee on Antimicrobial Susceptibility Testing.

Reading guide for broth microdilution. Version 4.0, 2022 (see: http://www.eucast.org).

Sensititre™ FINMER meropenem 64-0.03 μg/ml plates (Thermo Fischer Diagnostics custom plates; YFINMER) were modified by adding avibactam to rows 3 to 8 in concentrations of 64, 16, 8, 4, 1 and 0.25 μg/ml, respectively. The zinc chelator was added to the wells of the plate, where applicable. Three plates were made per experiment, one without the zinc chelator (plate 1), one with 16 μg/ml of the zinc chelator (plate 2) and one with 32 μg/ml of the zinc chelator (plate 3). The first row in all plates was used as a positive control for meropenem only, and the second row included the zinc chelator in addition to meropenem (no avibactam added to rows 1 or 2). The zinc chelator was added at the indicated concentration to rows 3-8 of plates 2 and 3. After incubation, assay plates were assessed visually, and the minimum inhibitory concentration (MIC) was determined as the lowest concentration of antimicrobial agent that completely inhibited bacterial growth as detected by the unaided eye.

### Results

The effect of the combination of zinc chelator/MEM/avibactam was tested on a clinically isolated Klebsiella pneumoniae Kpn St147 actively constitutionally expressing the two resistance mechanisms NDM-1 and KPC-2 simultaneously. As a negative control, bacteria+meropenem was tested alone. As a positive control, bacteria+MEM+zinc chelator was used. In Table 1 and 2, concentrations (μg/ml) of meropenem are given in the top row left to right and avibactam concentrations (μg/ml) are given top to bottom in column 1. An “X” indicates that the growth of the bacteria culture was not inhibited. A blank cell indicates no growth.

TABLE 1
Without zinc chelator. 16 μg/ml is present in the wells.

Meropenem (MEM, μg/mL)

| Negative | X | X | X | X | X | X | X | X | X | X | X | X |
|---|---|---|---|---|---|---|---|---|---|---|---|---|
| Avibactam | 64 | 32 | 16 | 8 | 4 | 2 | 1 | 0.5 | 0.25 | 0.12 | 0.06 | 0.03 |
| (μg/mL) ↓ | | | | | | | | | | | | |
| 64 | | | | | X | X | X | X | X | X | X | X |
| 16 | | | | X | X | X | X | X | X | X | X | X |
| 4 | X | X | X | X | X | X | X | X | X | X | X | X |
| 1 | X | X | X | X | X | X | X | X | X | X | X | X |
| 0.25 | X | X | X | X | X | X | X | X | X | X | X | X |
| 0.06 | X | X | X | X | X | X | X | X | X | X | X | X |

TABLE 2
With zinc chelator. 16 μg/ml is present in the wells.

Meropenem (MEM, μg/mL)

| Negative | X | X | X | X | X | X | X | X | X | X | X | X |
|---|---|---|---|---|---|---|---|---|---|---|---|---|
| Positive | X | X | X | X | X | X | X | X | X | X | X | X |
| Avibactam | 64 | 32 | 16 | 8 | 4 | 2 | 1 | 0.5 | 0.25 | 0.12 | 0.06 | 0.03 |
| (μg/mL) ↓ | | | | | | | | | | | | |
| 64 | | | | | | | | | | | | |
| 16 | | | | | | | | | | | | X |
| 8 | | | | | | | | | | | | X |
| 4 | | | | | | | | | | X | X | X |
| 1 | | | | | | | | | | X | X | X |
| 0.25 | | | | | | | | | X | X | X | X |

In Tables 1 and 2, the grey cells indicate values at MIC, i.e. the border between growth/no growth. With Klebsiella pneumoniae Kpn St147 actively constitutionally expressing the two resistance mechanisms NDM-1 and KPC-2, the presence of the zinc chelator at 16 μg/ml amplified the inhibitory effect by about 64-fold.

The fractional inhibitory concentration (FIC) is defined as the concentration that kills when used in combination with another agent divided by the concentration that has the same effect when used alone. The FIC index (FICI) for the combination of two agents is the sum of their individual FIC values. By convention, the FIC values of the most effective combination are used in calculating the FICI. The European Committee for Antimicrobial Susceptibility Testing (EUCAST) of the European Society of Clinical Microbiology and Infectious Diseases (ESCMID) 2000 adopts the following definitions to determine the susceptibility of bacteria to an antimicrobial agent (see EUCAST Definitive Document E. Def 1.2: Terminology relating to methods for the determination of susceptibility of bacteria to antimicrobial agents. Clin. Microbiol. Infect. 2000; 6(9): 503-508):
FICI value≤0.5; synergy
0.5<FICI value≤1; additive effect
1<FICI value <2; indifferent effect
FICI ≥2; antagonistic effect

To test for synergy, all data were subjected to the following formula in which A=zinc chelator; B=meropenem; and C=avibactam:

Σ FICI=MIC_{A}(combination)/MIC_{A}(alone)+MIC_{B}(combination)/MIC_{B}(alone)+MIC_{C}(combination)/MIC_{C}(alone)

TABLE 3
Calculated synergy based on the data from Table 2
MEM/Zinc Chelator/Avibactam

| | | Conc. | | | | Σ FICI |
|---|---|---|---|---|---|---|
| Conc. | | Zinc | FICI | Conc. | | If <0.5: |
| MEM | FICI | Chelator | Zinc | Avibactam | FICI | = |
| (μg/mL) | MEM | (μg/mL) | Chelator | (μg/mL) | Avibactam | synergy |
| 0.5 | 0.0039 | 16 | 0.008 | 0.25 | 0.0001 | 0.012 |
| 0.25 | 0.0020 | 16 | 0.008 | 1 | 0.0005 | 0.010 |
| 0.25 | 0.0020 | 16 | 0.008 | 4 | 0.0020 | 0.012 |
| 0.06 | 0.0005 | 16 | 0.008 | 8 | 0.0039 | 0.012 |
| 0.5 | 0.0039 | 32 | 0.016 | 0.25 | 0.0001 | 0.020 |
| 0.12 | 0.0009 | 32 | 0.016 | 1 | 0.0005 | 0.017 |
| 0.06 | 0.0005 | 32 | 0.016 | 4 | 0.0020 | 0.018 |
| 0.06 | 0.0005 | 32 | 0.016 | 8 | 0.0039 | 0.020 |

The effect of this combination was similarly strong on strains harbouring other resistance enzymes, e.g. Klebsiella pneumoniae ST101 (OXA-48), Klebsiella pneumoniae K66-45 (NDM-1) and Klebsiella pneumoniae BAA1705 (KPC-2).

### Examples 2 to 30—Minimal Inhibitory Concentration (MIC) Determination for a β-Lactam Antibiotic (Penicillin, Monobactam or Carbapenem) in Combination with a Zinc Chelator and a Serine β-Lactamase Inhibitor (sBLI) (Checkerboard Assays)

### Materials and Methods

The antibiotics and serine β-lactamase-inhibitors (sBLIs) were purchased from the following suppliers: Meropenem (MEM): Adom Bioscience; Piperacillin (PIP): Sigma-Aldrich; Amoxicillin (AMOX): Sigma-Aldrich; Ampicillin (AMP): Sigma-Aldrich Aztreonam (AZT): Sigma-Aldrich; Sulbactam (SUL): Sigma-Aldrich; Avibactam (AVI): Biosynth; Vaborbactam (VAB): ABCR; Tazobactam (TAZ): Sigma-Aldrich.

In each of Examples 2-30, the compound of Example 26 of WO 2018/033719 was employed as the zinc chelator. The following abbreviations are used in these examples: sBLI means serine-β-lactamase inhibitor; CFU means colony-forming unit, which is a group of microbes that grows from a single cell on a petri plate; DMSO means dimethyl sulfoxide; MHB means Mueller-Hinton broth; caMHB means cation-adjusted Mueller-Hinton broth; MIC means minimum inhibitory concentration—the lower the MIC, the more efficient the combination against the respective bacterial strain.

All examples demonstrated synergy between the zinc chelator and the other two components in the triple combination, meaning that the MIC for the double combination of the antibiotic drug and the serine-β-lactamase inhibitor alone was always higher in one or more wells than the triple combination of the zinc chelator+the antibiotic drug+the serine-β-lactamase inhibitor. Since neither the zinc chelator nor the sBLI have intrinsic antibiotic effect, the MIC value always means the MIC of the antibiotic component in the combination. Thus, in most of the experiments, a Fractional Inhibitory Concentration Index (FICI) could not be calculated, and the conclusion of synergy was made through visual reading of the bacterial growth in the wells in the combination experiment.

### General Protocol Used in Examples 2-7 Employing a Fixed Ratio of sBLI to Antibiotic within Each Experiment

Antimicrobial susceptibility testing (AST) by broth microdilution (MIC determination) was performed according to The European Committee on Antimicrobial Susceptibility Testing (EUCAST) guidelines (see Reading guide for broth microdilution Version 5.0, 2024: https://www.eucast.org). Care was taken that a final bacterial inoculum of 3-7×10⁵ CFU/mL was used for each test condition (each well) and that final concentrations in each well of solvents used for making stock solutions of chemicals did not exceed concentrations having an effect on bacterial growth (e.g. DMSO).

Sterile clear round bottom polystyrene (non-treated) 96-well microplates (Corning 3788) were used for making serial two-fold dilutions of the antibiotic and the sBLI in cation-adjusted Mueller Hinton II broth (caMHB; Thermo Scientific, T3462, containing 29.4 μM Zn²⁺), across the columns of the microtiter plate (256-0.06 μg/mL; columns 1-12). The checkerboard plates were all made by keeping the sBLI at a fixed ratio relative to antibiotic (absolute concentrations of each, in μg/mL) to all wells containing sample in each plate. Additionally, a set of samples was included in each test plate, where the zinc chelator was added, at various fixed concentrations, of 16 μg/mL, 8 μg/mL and/or 4 μg/mL (as specified in each Example). All sample wells contained growth medium (caMHB), and inoculated bacteria at a final inoculum between 3-7×10⁵ CFU/mL. After incubation at 37° C. for 20 h, assay plates were assessed visually, and the minimum inhibitory concentration (MIC) was determined as the lowest concentration of antimicrobial agent that completely inhibited bacterial growth as detected by the unaided eye. Synergy for the triple combination (antibiotic, sBLI, and zinc chelator) was defined as when the MIC in rows containing the zinc chelator in combination with antibiotic and sBLI (triple combination), was lower than for rows containing antibiotic and sBLI (double combination; no zinc chelator).

### General Protocol 2 Used in Examples 8-30 Employing a Fixed Concentration of sBLI within Each Experiment

Antimicrobial susceptibility testing (AST) by broth microdilution (MIC determination) was performed according to The European Committee on Antimicrobial Susceptibility Testing (EUCAST) guidelines (see Reading guide for broth microdilution Version 5.0, 2024: https://www.eucast.org). Care was taken that a final bacterial inoculum of 3-7×10⁵ CFU/mL was used for each test condition (each well) and that final concentrations in each well of solvents used for making stock solutions of chemicals did not exceed concentrations having an effect on bacterial growth (e.g. DMSO).

For combinations containing meropenem as the antibiotic, Sensititre™ FINMER meropenem 64-0.03 μg/mL plates (Thermo Fischer Diagnostics custom plates; YFINMER) were used in most of the experiments. For other antibiotics (and for meropenem whenever a higher start concentration than 64 μg/mL was needed) sterile clear round bottom polystyrene (non-treated) 96-well microplates (Corning 3788) were used for making serial two-fold dilutions of the antibiotic in cation-adjusted Mueller Hinton II broth (caMHB; Thermo Scientific, T3462, containing 29.4 μM Zn²⁺ or MILLIPORE, 90922-500G, containing 5.6 μM Zn²⁺), across the columns of the microtiter plate (128-0.06 μg/mL; columns 1-12).

The checkerboard plates were all modified by adding a serine β-lactamase inhibitor (sBLI) at a fixed concentration of 16 μg/mL. Additionally, the zinc chelator was added at various fixed concentrations. Samples were mixed so that all plates contained a control containing antibiotic alone (no sBLI, no zinc chelator), a sample containing antibiotic and sBLI (double combination; no zinc chelator), and a sample containing antibiotic, sBLI and zinc chelator (triple combination). All test wells used in each plate contained growth medium (caMHB), and inoculated bacteria at a final inoculum between 3-7×10⁵ CFU/mL. After incubation at 37° C. for 20 h, assay plates were assessed visually, and the minimum inhibitory concentration (MIC) was determined as the lowest concentration of antimicrobial agent that completely inhibited bacterial growth as detected by the unaided eye. Synergy for the triple combination (antibiotic, sBLI, and zinc chelator) was defined as when the MIC in any of the rows containing the zinc chelator in combination with antibiotic and sBLI (triple combination), was lower than for antibiotic alone and lower than for the antibiotic and sBLI double combination.

### Example 2—Minimal Inhibitory Concentration (MIC) Determination of Meropenem (MEM) in Combination with Zinc Chelator and Avibactam (AVI) Vs. Klebsiella pneumoniae K66-45

The effect of the triple combination of the zinc chelator with MEM and AVI was tested on a clinical isolate of Klebsiella pneumoniae, strain K66-45, carrying genes encoding β-lactamase NDM-1 (Table 4). Synergy with the zinc chelator was observed at concentrations of 16 μg/mL and 8 μg/mL.

Experimental results from MIC determination with a triple combination of MEM, AVI and the zinc chelator are given in Table 4. Two technical replicates were performed for each sample. An X in rows 1-8 in the table indicates bacterial growth, while a blank cell indicates no growth. Concentrations (μg/mL) of MEM are given in row 0a. Concentrations (μg/mL) of AVI are given in row Ob. The zinc chelator was added in fixed concentrations, at 16 μg/mL in rows 3 and 4, at 8 μg/mL in rows 5 and 6, and at 4 μg/mL in rows 7 and 8. As a control, the effect of MEM and AVI (no zinc chelator) was tested (rows 1 and 2). All wells contained bacterial growth medium (caMHB, 29.4 μM Zn²⁺) and were inoculated with the test bacterium. The grey cells indicate the meropenem MIC.

### Example 3—Minimal Inhibitory Concentration (MIC) Determination of Meropenem (MEM) in Combination with Zinc Chelator and Sulbactam (SUL) Vs. Klebsiella pneumoniae K66-45

The effect of the triple combination of the zinc chelator with MEM and SUL was tested on a clinical isolate of Klebsiella pneumoniae, strain K66-45, carrying genes encoding β-lactamase NDM-1 (Table 5). Synergy with the zinc chelator was observed at concentrations of 16 μg/mL, 8 μg/mL and 4 μg/mL.

Experimental results from MIC determination with a triple combination of MEM, SUL and the zinc chelator are given in Table 5. Two technical replicates were performed for each sample. An X in rows 1-8 in the table indicates bacterial growth, while a blank cell indicates no growth. Concentrations (μg/mL) of MEM are given in row 0a. Concentrations (μg/mL) of SUL are given in row Ob. The zinc chelator was added in fixed concentrations, at 16 μg/mL in rows 3 and 4, at 8 μg/mL in rows 5 and 6, and 4 μg/mL in rows 7 and 8. As a control, the effect of MEM and SUL (no zinc chelator) was tested (rows 1 and 2). All wells contained bacterial growth medium (caMHB, 29.4 μM Zn²⁺) and were inoculated with the test bacterium. The grey cells indicate the meropenem MIC.

### Example 4—Minimal Inhibitory Concentration (MIC) Determination of Meropenem (MEM) in Combination with Zinc Chelator and Tazobactam (TAZ) Vs. Klebsiella pneumoniae K66-45

The effect of the triple combination of the zinc chelator with MEM and TAZ was tested on a clinical isolate of Klebsiella pneumoniae, strain K66-45, carrying genes encoding β-lactamase NDM-1 (Table 6). Synergy with the zinc chelator was observed at concentrations of 16 μg/mL, 8 μg/mL and 4 μg/mL

Experimental results from MIC determination with a triple combination of MEM, TAZ and the zinc chelator are given in Table 6. Two technical replicates were performed for each sample. An X in rows 1-8 in the table indicates bacterial growth, while a blank cell indicates no growth. Concentrations (μg/mL) of MEM are given in row 0a. Concentrations (μg/mL) of TAZ are given in row Ob. The zinc chelator was added in fixed concentrations, at 16 μg/mL in rows 3 and 4, at 8 μg/mL in rows 5 and 6, and 4 μg/mL in rows 7 and 8. As a control, the effect of MEM and TAZ (no zinc chelator) was tested (rows 1 and 2). All wells contained bacterial growth medium (caMHB, 29.4 μM Zn²⁺) and were inoculated with the test bacterium. The grey cells indicate the meropenem MIC.

### Example 5—Minimal Inhibitory Concentration (MIC) Determination of Meropenem (MEM) in Combination with Zinc Chelator and Sulbactam (SUL) Vs. Klebsiella pneumoniae ST147

The effect of the triple combination of the zinc chelator with MEM and SUL was tested on a clinical isolate of Klebsiella pneumoniae ST147, carrying genes encoding β-lactamases NDM-1 and KPC-2 (Table 7). Synergy with the zinc chelator was observed at concentrations of 16 μg/mL, 8 μg/mL and 4 μg/mL

Experimental results from MIC determination with a triple combination of MEM, SUL and the zinc chelator are given in Table 7. Two technical replicates were performed for each sample. An X in rows 1-8 in the table indicates bacterial growth, while a blank cell indicates no growth. Concentrations (μg/mL) of MEM are given in row 0a. Concentrations (μg/mL) of SUL are given in row Ob. The zinc chelator was added in fixed concentrations, at 16 μg/mL in rows 3 and 4, at 8 μg/mL in rows 5 and 6, and 4 μg/mL in rows 7 and 8. As a control, the effect of MEM and SUL (no zinc chelator) was tested (rows 1 and 2). All wells contained bacterial growth medium (caMHB, 29.4 μM Zn²⁺) and were inoculated with the test bacterium. The grey cells indicate the meropenem MIC. For rows where the exact MIC could not be determined, the MIC was set at >256 μg/mL (black shading) or <0.12 μg/mL (light grey shading), respectively.

### Example 6—Minimal Inhibitory Concentration (MIC) Determination of Meropenem (MEM) in Combination with Zinc Chelator and Tazobactam (TAZ) Vs. Klebsiella pneumoniae ST147

The effect of the triple combination of the zinc chelator with MEM and TAZ was tested on a clinical isolate of Klebsiella pneumoniae ST147, carrying genes encoding β-lactamases NDM-1 and KPC-2 (Table 8). Synergy with the zinc chelator was observed at concentrations of 16 μg/mL, 8 μg/mL and 4 μg/mL.

Experimental results from MIC determination with a triple combination of MEM, TAZ and the zinc chelator are given in Table 8. Two technical replicates were performed for each sample. An X in rows 1-8 in the table indicates bacterial growth, while a blank cell indicates no growth. Concentrations (μg/mL) of MEM are given in row 0a. Concentrations (μg/mL) of TAZ are given in row Ob. The zinc chelator was added in fixed concentrations, at 16 μg/mL in rows 3 and 4, at 8 μg/mL in rows 5 and 6, and 4 μg/mL in rows 7 and 8. As a control, the effect of MEM and TAZ (no zinc chelator) was tested (rows 1 and 2). All wells contained bacterial growth medium (caMHB, 29.4 μM Zn²⁺) and were inoculated with the test bacterium. The grey cells indicate the meropenem MIC. For rows where the exact MIC could not be determined, the MIC was set at >256 μg/mL (black shading) or <0.12 μg/mL (light grey shading), respectively.

### Example 7—Minimal Inhibitory Concentration (MIC) Determination of Meropenem (MEM) in Combination with Zinc Chelator and Avibactam (AVI) Vs. Klebsiella pneumoniae ST147

The effect of the triple combination of the zinc chelator with MEM and AVI was tested on a clinical isolate of Klebsiella pneumoniae ST147, carrying genes encoding β-lactamases NDM-1 and KPC-2 (Table 9). Synergy with the zinc chelator was observed at concentrations of 16 μg/mL and 8 μg/mL.

Experimental results from MIC determination with a triple combination of MEM, AVI and the zinc chelator are given in Table 9. Two technical replicates were performed for each sample. An X in rows 1-8 in the table indicates bacterial growth, while a blank cell indicates no growth. Concentrations (μg/mL) of MEM are given in row 0a. Concentrations (μg/mL) of AVI are given in row Ob. The zinc chelator was added in fixed concentrations, at 16 μg/mL in rows 3 and 4, at 8 μg/mL in rows 5 and 6, and 4 μg/mL in rows 7 and 8. As a control, the effect of MEM and AVI (no zinc chelator) was tested (rows 1 and 2). All wells contained bacterial growth medium (caMHB, 29.4 μM Zn²⁺) and were inoculated with the test bacterium. The grey cells indicate the meropenem MIC.

### Example 8—Minimal Inhibitory Concentration (MIC) Determination of Piperacillin (PIP) in Combination with Zinc Chelator and Tazobactam (TAZ) Vs. Klebsiella pneumoniae K66-45

The effect of the triple combination of the zinc chelator with PIP and TAZ was tested on a clinical isolate of Klebsiella pneumoniae, strain K66-45, carrying genes encoding the β-lactamase NDM-1 (Table 10). Synergy with the zinc chelator was observed at concentrations of 16 μg/mL, 8 μg/mL, and 4 μg/mL.

Experimental results from MIC determination with a triple combination of PIP, TAZ and the zinc chelator are given in Table 10. An X in rows 1-5 in the table indicates growth of the bacterial inoculum, while a blank cell indicates no growth. Concentrations (μg/mL) of PIP are given in row 0. TAZ and the zinc chelator were added in fixed concentrations (TAZ at 16 μg/mL in rows 2-5, zinc chelator at 16 μg/mL in row 3, at 8 μg/mL in row 4, and at 4 μg/mL in row 5). As a control, the effect of PIP alone was tested (row 1). All wells contained bacterial growth medium (caMHB, 5.6 μM Zn²⁺) and were inoculated with the test bacterium. The grey cells indicate the piperacillin MIC. For rows where the exact MIC could not be determined, the MIC was set at >128 μg/mL (black shading) or <0.06 μg/mL (light grey shading), respectively.

### Example 9—Minimal Inhibitory Concentration (MIC) Determination of Piperacillin (PIP) in Combination with Zinc Chelator and Tazobactam (TAZ) Vs. Klebsiella pneumoniae K66-45

The effect of the triple combination of the zinc chelator with PIP and TAZ was tested on a clinical isolate of Klebsiella pneumoniae strain K66-45 carrying genes encoding the β-lactamase NDM-1 (Table 11). Synergy with the zinc chelator was observed at a concentration of 16 μg/mL.

Experimental results from MIC determination with a triple combination of PIP, TAZ and the zinc chelator are given in Table 11. An X in rows 1~4 in the table indicates growth of the bacterial inoculum, while a blank cell indicates no growth. Concentrations (μg/mL) of PIP are given in row 0. TAZ and the zinc chelator were added in fixed concentrations (TAZ at 16 μg/mL in rows 2-4, zinc chelator at 16 μg/mL in row 3 and at 8 μg/mL in row 4). As a control, the effect of PIP alone was tested (row 1). All wells contained bacterial growth medium (caMHB, 29.4 μM Zn²⁺) and were inoculated with the test bacterium. The grey cells indicate the piperacillin MIC. For rows where the exact MIC could not be determined, the MIC was set at >128 μg/mL (black shading) or <0.06 μg/mL (light grey shading), respectively.

### Example 10—Minimal Inhibitory Concentration (MIC) Determination of Ampicillin (AMP) in Combination with Zinc Chelator and Sulbactam (SUL) Vs. Acinetobacter baumannii ST25

The effect of the triple combination of the zinc chelator with AMP and SUL was tested on a clinical isolate of Acinetobacter baumannii ST 25 carrying genes encoding the β-lactamases NDM-1, OXA-58, OXA-64, OXA-10, VEB-21 and ADC-26 (Table 12). Synergy with the zinc chelator was observed at a concentration of 16 μg/mL.

Experimental results from MIC determination with a triple combination of AMP, SUL and the zinc chelator are given in Table 12. Two technical replicates were performed for each sample. An X in rows 1-8 in the table indicates growth of the bacterial inoculum, while a blank cell indicates no growth. Concentrations (μg/mL) of AMP are given in row 0. SUL and the zinc chelator were added in fixed concentrations (SUL at 16 μg/mL in rows 3-8, zinc chelator at 16 μg/mL in rows 5-6 and at 8 μg/mL in rows 7-8). As a control, the effect of AMP alone was tested (rows 1-2). All wells contained bacterial growth medium (caMHB, 29.4 μM Zn²⁺) and were inoculated with the test bacterium. The grey cells indicate the ampicillin MIC. For rows where the exact MIC could not be determined, the MIC was set at >128 μg/ml (black shading) or <0.06 μg/mL (light grey shading), respectively.

### Example 11—Minimal Inhibitory Concentration (MIC) Determination of Meropenem (MEM) in Combination with Zinc Chelator and Sulbactam (SUL) Vs. Acinetobacter baumannii ST15

The effect of the triple combination of the zinc chelator with MEM and SUL was tested on a clinical isolate of Acinetobacter baumannii ST15 carrying genes encoding the β-lactamases NDM-1 OXA-51, ADC-263 (Table 13). Synergy with the zinc chelator was observed at a concentration of 16 μg/mL.

Experimental results from MIC determination with a triple combination of MEM, SUL and the zinc chelator are given in Table 13. An X in rows 1~4 in the table indicates growth of the bacterial inoculum, while a blank cell indicates no growth. Concentrations (μg/mL) of MEM are given in row 0. SUL and the zinc chelator were added in fixed concentrations (SUL at 16 μg/mL in rows 2-4, zinc chelator at 16 μg/mL in row 3 and at 8 μg/mL in row 4). As a control, the effect of MEM alone was tested (row 1). All wells contained bacterial growth medium (caMHB, 29.4 μM Zn²⁺) and were inoculated with the test bacterium. The grey cells indicate the meropenem MIC. For rows where the exact MIC could not be determined, the MIC was set at >128 μg/mL (black shading) or <0.06 μg/mL (light grey shading), respectively.

### Example 12—Minimal Inhibitory Concentration (MIC) Determination of Amoxicillin (AMOX) in Combination with Zinc Chelator and Sulbactam (SUL) Vs. Acinetobacter baumannii ST15

The effect of the triple combination of the zinc chelator with AMOX and SUL was tested on a clinical isolate of Acinetobacter baumannii ST15 carrying genes encoding the β-lactamases NDM-1 OXA-51, ADC-263 (Table 14). Synergy with the zinc chelator was observed at a concentration of 16 μg/mL.

Experimental results from MIC determination with a triple combination of AMOX, SUL and the zinc chelator are given in Table 14. An X in rows 1~4 in the table indicates growth of the bacterial inoculum, while a blank cell indicates no growth. Concentrations (μg/mL) of AMOX are given in row 0. SUL and the zinc chelator were added in fixed concentrations (SUL at 16 μg/mL in rows 2-4, zinc chelator at 16 μg/mL in row 3 and at 8 μg/mL in row 4). As a control, the effect of AMOX alone was tested (row 1). All wells contained bacterial growth medium (caMHB, 29.4 μM Zn²⁺) and were inoculated with the test bacterium. The grey cells indicate the amoxicillin MIC. For rows where the exact MIC could not be determined, the MIC was set at >128 μg/mL (black shading) or <0.06 μg/mL (light grey shading), respectively.

### Example 13—Minimal Inhibitory Concentration (MIC) Determination of Ampicillin (AMP) in Combination with Zinc Chelator and Sulbactam (SUL) Vs. Acinetobacter baumannii ST15

The effect of the triple combination of the zinc chelator with AMP and SUL was tested on a clinical isolate of Acinetobacter baumannii ST15 carrying genes encoding the β-lactamases NDM-1 OXA-51, ADC-263 (Table 15). Synergy with the zinc chelator was observed at a concentration of 16 μg/mL.

Experimental results from MIC determination with a triple combination of AMP, SUL and the zinc chelator are given in Table 15. Two technical replicates were performed for each sample. An X in rows 1-8 in the table indicates growth of the bacterial inoculum, while a blank cell indicates no growth. Concentrations (μg/mL) of AMP are given in row 0. SUL and the zinc chelator were added in fixed concentrations (SUL at 16 μg/mL in rows 3-8, zinc chelator at 16 μg/mL in rows 5-6 and at 8 μg/mL in rows 7-8). As a control, the effect of AMP alone was tested (rows 1-2). All wells contained bacterial growth medium (caMHB, 29.4 μM Zn²⁺) and were inoculated with the test bacterium. The grey cells indicate the ampicillin MIC. For rows where the exact MIC could not be determined, the MIC was set at >128 μg/mL (black shading) or <0.06 μg/mL (light grey shading), respectively.

### Example 14—Minimal Inhibitory Concentration (MIC) Determination of Piperacillin (PIP) in Combination with Zinc Chelator and Tazobactam (TAZ) Vs. Acinetobacter baumannii ST15

The effect of the triple combination of the zinc chelator with PIP and TAZ was tested on a clinical isolate of Acinetobacter baumannii ST15 carrying genes encoding the β-lactamases NDM-1 OXA-51, ADC-263 (Table 16). Synergy with the zinc chelator was observed at concentrations of 16 μg/mL and 8 μg/mL.

Experimental results from MIC determination with a triple combination of PIP, TAZ and the zinc chelator are given in Table 16. An X in rows 1~4 in the table indicates growth of the bacterial inoculum, while a blank cell indicates no growth. Concentrations (μg/mL) of PIP are given in row 0. TAZ and the zinc chelator were added in fixed concentrations (TAZ at 16 μg/mL in rows 2-4, zinc chelator at 16 μg/mL in row 3 and at 8 μg/mL in row 4). As a control, the effect of PIP alone was tested (row 1). All wells contained bacterial growth medium (caMHB, 29.4 μM Zn²⁺) and were inoculated with the test bacterium. The grey cells indicate the piperacillin MIC. For rows where the exact MIC could not be determined, the MIC was set at >128 μg/mL (black shading) or <0.06 μg/mL (light grey shading).

### Example 15—Minimal Inhibitory Concentration (MIC) Determination of Meropenem (MEM) in Combination with Zinc Chelator and Tazobactam (TAZ) Vs. Acinetobacter baumannii ST15

The effect of the triple combination of the zinc chelator with MEM and TAZ was tested on a clinical isolate of Acinetobacter baumannii ST15 carrying genes encoding the β-lactamases NDM-1 OXA-51, ADC-263 (Table 17). Synergy with the zinc chelator was observed at concentrations of 16 μg/mL 8 μg/mL and 4 μg/mL.

Experimental results from MIC determination with a triple combination of MEM, TAZ and the zinc chelator are given in Table 17. An X in rows 1-5 in the table indicates growth of the bacterial inoculum, while a blank cell indicates no growth. Concentrations (μg/mL) of MEM are given in row 0. TAZ and the zinc chelator were added in fixed concentrations (TAZ at 16 μg/mL in rows 2-5, zinc chelator at 16 μg/mL in row 3, at 8 μg/mL in row 4, and at 4 μg/mL in row 5). As a control, the effect of MEM alone was tested (row 1). All wells contained bacterial growth medium (caMHB, 5.6 μM Zn²⁺) and were inoculated with the test bacterium. The grey cells indicate the meropenem MIC.

### Example 16—Minimal Inhibitory Concentration (MIC) Determination of Amoxicillin (AMOX) in Combination with Zinc Chelator and Sulbactam (SUL) Vs. Acinetobacter baumannii ST15

The effect of the triple combination of the zinc chelator with AMOX and SUL was tested on a clinical isolate of Acinetobacter baumannii ST15 carrying genes encoding the β-lactamases NDM-1 OXA-51, ADC-263 (Table 18). Synergy with the zinc chelator was observed at concentrations of 16 μg/mL, 8 μg/mL, and 4 μg/mL.

Experimental results from MIC determination with a triple combination of AMOX, SUL and the zinc chelator are given in Table 18. An X in rows 1-5 in the table indicates growth of the bacterial inoculum, while a blank cell indicates no growth. Concentrations (μg/mL) of AMOX are given in row 0. SUL and the zinc chelator were added in fixed concentrations (SUL at 16 μg/mL in rows 2-5, zinc chelator at 16 μg/mL in row 3, at 8 μg/mL in row 4, and at 4 μg/mL in row 5). As a control, the effect of AMOX alone was tested (row 1). All wells contained bacterial growth medium (caMHB, 5.6 μM Zn²⁺) and were inoculated with the test bacterium. The grey cells indicate the amoxicillin MIC. For rows where the exact MIC could not be determined, the MIC was set at >128 μg/mL (black shading) or <0.06 μg/mL (light grey shading).

### Example 17—Minimal Inhibitory Concentration (MIC) Determination of Ampicillin (AMP) in Combination with Zinc Chelator and Sulbactam (SUL) Vs. Acinetobacter baumannii ST15

The effect of the triple combination of the zinc chelator with AMP and SUL was tested on a clinical isolate of Acinetobacter baumannii ST15 carrying genes encoding the β-lactamases NDM-1 OXA-51, ADC-263 (Table 19). Synergy with the zinc chelator was observed at concentrations of 16 μg/mL, 8 μg/mL, and 4 μg/mL.

Experimental results from MIC determination with a triple combination of AMP, SUL and the zinc chelator are given in Table 19. An X in rows 1-5 in the table indicates growth of the bacterial inoculum, while a blank cell indicates no growth. Concentrations (μg/mL) of AMP are given in row 0. SUL and the zinc chelator were added in fixed concentrations (SUL at 16 μg/mL in rows 2-5, zinc chelator at 16 μg/mL in row 3, at 8 μg/mL in row 4, and at 4 μg/mL in row 5). As a control, the effect of AMP alone was tested (row 1). All wells contained bacterial growth medium (caMHB, 5.6 μM Zn²⁺) and were inoculated with the test bacterium. The grey cells indicate the ampicillin MIC. For rows where the exact MIC could not be determined, the MIC was set at >128 μg/mL (black shading) or <0.06 μg/mL (light grey shading).

### Example 18—Minimal Inhibitory Concentration (MIC) Determination of Piperacillin (PIP) in Combination with Zinc Chelator and Tazobactam (TAZ) Vs. Acinetobacter baumannii ST15

The effect of the triple combination of the zinc chelator with PIP and TAZ was tested on a clinical isolate of Acinetobacter baumannii ST15 carrying genes encoding the β-lactamases NDM-1 OXA-51, ADC-263 (Table 20). Synergy with the zinc chelator was observed at concentrations of 16 μg/mL and 8 μg/mL.

Experimental results from MIC determination with a triple combination of PIP, TAZ and the zinc chelator are given in Table 20. An X in rows 1-5 in the table indicates growth of the bacterial inoculum, while a blank cell indicates no growth. Concentrations (μg/mL) of PIP are given in row 0. TAZ and the zinc chelator were added in fixed concentrations (TAZ at 16 μg/mL in rows 2-5, zinc chelator at 16 μg/mL in row 3, at 8 μg/mL in row 4, and at 4 μg/mL in row 5). As a control, the effect of PIP alone was tested (row 1). All wells contained bacterial growth medium (caMHB, 5.6 μM Zn²⁺) and were inoculated with the test bacterium. The grey cells indicate the piperacillin MIC. For rows where the exact MIC could not be determined, the MIC was set at >128 μg/mL (black shading) or <0.06 μg/mL (light grey shading).

### Example 19—Minimal Inhibitory Concentration (MIC) Determination of Meropenem (MEM) in Combination with Zinc Chelator and Tazobactam (TAZ) Vs. Pseudomonas aeruginosa ST773

The effect of the triple combination of the zinc chelator with MEM and TAZ was tested on a clinical isolate of Pseudomonas aeruginosa ST773 carrying genes encoding the β-lactamases NDM-1, OXA-395, PDC-16 (Table 21). Synergy with the zinc chelator was observed at a concentration of 16 μg/mL.

Experimental results from MIC determination with a triple combination of MEM, TAZ and the zinc chelator are given in Table 21. An X in rows 1~4 in the table indicates growth of the bacterial inoculum, while a blank cell indicates no growth. Concentrations (μg/mL) of MEM are given in row 0. TAZ and the zinc chelator were added in fixed concentrations (TAZ at 16 μg/mL in rows 2-4, zinc chelator at 16 μg/mL in row 3 and at 8 μg/mL in row 4). As a control, the effect of MEM alone was tested (row 1). All wells contained bacterial growth medium (caMHB, 29.4 μM Zn²⁺) and were inoculated with the test bacterium. The grey cells indicate the meropenem MIC. For rows where the exact MIC could not be determined, the MIC was set at >128 μg/mL (black shading) or <0.06 μg/mL (light grey shading).

### Example 20—Minimal Inhibitory Concentration (MIC) Determination of Aztreonam (AZT) in Combination with Zinc Chelator and Avibactam (AVI) Vs. Pseudomonas aeruginosa ST773

The effect of the triple combination of the zinc chelator with AZT and AVI was tested on a clinical isolate of Pseudomonas aeruginosa ST773 carrying genes encoding the β-lactamases NDM-1, OXA-395, PDC-16 (Table 22). Synergy with the zinc chelator was observed at a concentration of 16 μg/mL.

Experimental results from MIC determination with a triple combination of AZT, AVI and the zinc chelator are given in Table 22. An X in rows 1~4 in the table indicates growth of the bacterial inoculum, while a blank cell indicates no growth. Concentrations (μg/mL) of AZT are given in row 0. AVI and the zinc chelator were added in fixed concentrations (AVI at 16 μg/mL in rows 2-4, zinc chelator at 16 μg/mL in row 3 and at 8 μg/mL in row 4). As a control, the effect of AZT alone was tested (row 1). All wells contained bacterial growth medium (caMHB, 29.4 μM Zn²⁺) and were inoculated with the test bacterium. The grey cells indicate the aztreonam MIC.

### Example 21—Minimal Inhibitory Concentration (MIC) Determination of Piperacillin (PIP) in Combination with Zinc Chelator and Avibactam (AVI) Vs. Pseudomonas aeruginosa ST773

The effect of the triple combination of the zinc chelator with PIP and AVI was tested on a clinical isolate of Pseudomonas aeruginosa ST773 carrying genes encoding the β-lactamases NDM-1, OXA-395, PDC-16 (Table 23). Synergy with the zinc chelator was observed at concentrations of 16 μg/mL and at 8 μg/mL for one replicate. The results for one replicate of the concentration 8 μg/mL was not interpretable according to EUCAST guidelines.

Experimental results from MIC determination with a triple combination of PIP, AVI and the zinc chelator are given in Table 23. Two technical replicates were performed for each sample. An X in rows 1-8 in the table indicates growth of the bacterial inoculum, while a blank cell indicates no growth. Concentrations (μg/mL) of PIP are given in row 0. AVI and the zinc chelator were added in fixed concentrations (AVI at 16 μg/mL in rows 3-8, zinc chelator at 16 μg/mL in rows 5-6 and at 8 μg/mL in rows 7-8). As a control, the effect of PIP alone was tested (rows 1-2). All wells contained bacterial growth medium (caMHB, 29.4 μM Zn²⁺) and were inoculated with the test bacterium. The grey cells indicate the piperacillin MIC. For rows where the exact MIC could not be determined, the MIC was set at >128 μg/mL (black shading) or <0.06 μg/mL (light grey shading).

### Example 22—Minimal Inhibitory Concentration (MIC) Determination of Piperacillin (PIP) in Combination with Zinc Chelator and Tazobactam (TAZ) Vs. Pseudomonas aeruginosa ST773

The effect of the triple combination of the zinc chelator with PIP and TAZ was tested on a clinical isolate of Pseudomonas aeruginosa ST773 carrying genes encoding the β-lactamases NDM-1, OXA-395, PDC-16 (Table 24). Synergy with the zinc chelator was observed at a concentration of 16 μg/mL.

Experimental results from MIC determination with a triple combination of PIP, TAZ and the zinc chelator are given in Table 24. An X in rows 1~4 in the table indicates growth of the bacterial inoculum, while a blank cell indicates no growth. Concentrations (μg/mL) of PIP are given in row 0. TAZ and the zinc chelator were added in fixed concentrations (TAZ at 16 μg/mL in rows 2-4, zinc chelator at 16 μg/mL in row 3 and at 8 μg/mL in row 4). As a control, the effect of PIP alone was tested (row 1). All wells contained bacterial growth medium (caMHB, 29.4 μM Zn²⁺) and were inoculated with the test bacterium. The grey cells indicate the piperacillin MIC. For rows where the exact MIC could not be determined, the MIC was set at >128 μg/mL (black shading) or <0.06 μg/mL (light grey shading).

### Example 23—Minimal Inhibitory Concentration (MIC) Determination of Meropenem (MEM) in Combination with Zinc Chelator and Sulbactam (SUL) Vs. Pseudomonas aeruginosa ST773

The effect of the triple combination of the zinc chelator with MEM and SUL was tested on a clinical isolate of Pseudomonas aeruginosa ST773 carrying genes encoding the β-lactamases NDM-1, OXA-395, PDC-16 (Table 25). Synergy with the zinc chelator was observed at concentrations of 16 μg/mL, 8 μg/mL and 4 μg/mL. The result for the concentration of 4 μg/ml was not interpretable according to EUCAST guidelines.

Experimental results from MIC determination with a triple combination of MEM, SUL and the zinc chelator are given in Table 25. An X in rows 1-5 in the table indicates growth of the bacterial inoculum, while a blank cell indicates no growth. Concentrations (μg/mL) of MEM are given in row 0. SUL and the zinc chelator were added in fixed concentrations (SUL at 16 μg/mL in rows 2-5, zinc chelator at 16 μg/mL in row 3, at 8 μg/mL in row 4, and at 4 μg/mL in row 5). As a control, the effect of MEM alone was tested (row 1). All wells contained bacterial growth medium (caMHB, 5.6 μM Zn²⁺) and were inoculated with the test bacterium. The grey cells indicate the meropenem MIC. For rows where the exact MIC could not be determined, the MIC was set at >64 μg/mL (black shading) or <0.03 μg/mL (light grey shading).

### Example 24—Minimal Inhibitory Concentration (MIC) Determination of Meropenem (MEM) in Combination with Zinc Chelator and Tazobactam (TAZ) Vs. Pseudomonas aeruginosa ST773

The effect of the triple combination of the zinc chelator with MEM and TAZ was tested on a clinical isolate of Pseudomonas aeruginosa ST773 carrying genes encoding the β-lactamases NDM-1, OXA-395, PDC-16 (Table 26). Synergy with the zinc chelator was observed at a concentration of 8 μg/mL. The results for the concentrations of 16 μg/mL and 4 μg/ml were not interpretable according to EUCAST guidelines.

Experimental results from MIC determination with a triple combination of MEM, TAZ and the zinc chelator are given in Table 26. An X in rows 1-5 in the table indicates growth of the bacterial inoculum, while a blank cell indicates no growth. Concentrations (μg/mL) of MEM are given in row 0. TAZ and the zinc chelator were added in fixed concentrations (TAZ at 16 μg/mL in rows 2-5, zinc chelator at 16 μg/mL in row 3, at 8 μg/mL in row 4, and at 4 μg/mL in row 5). As a control, the effect of MEM alone was tested (row 1). All wells contained bacterial growth medium (caMHB, 5.6 μM Zn²⁺) and were inoculated with the test bacterium. The grey cells indicate the meropenem MIC. For rows where the exact MIC could not be determined, the MIC was set at >64 μg/mL (black shading) or <0.03 μg/mL (light grey shading).

### Example 25—Minimal Inhibitory Concentration (MIC) Determination of Piperacillin (PIP) in Combination with Zinc Chelator and Avibactam (AVI) Vs. Pseudomonas aeruginosa ST773

The effect of the triple combination of the zinc chelator with PIP and AVI was tested on a clinical isolate of Pseudomonas aeruginosa ST773 carrying genes encoding the β-lactamases NDM-1, OXA-395, PDC-16 (Table 27). Synergy with the zinc chelator was observed at concentrations of 16 μg/mL, 8 μg/mL and 4 μg/mL.

Experimental results from MIC determination with a triple combination of PIP, AVI and the zinc chelator are given in Table 27. An X in rows 1-5 in the table indicates growth of the bacterial inoculum, while a blank cell indicates no growth. Concentrations (μg/mL) of PIP are given in row 0. AVI and the zinc chelator were added in fixed concentrations (AVI at 16 μg/mL in rows 2-5, zinc chelator at 16 μg/mL in row 3, at 8 μg/mL in row 4, and at 4 μg/mL in row 5). As a control, the effect of PIP alone was tested (row 1). All wells contained bacterial growth medium (caMHB, 5.6 μM Zn²⁺) and were inoculated with the test bacterium. The grey cells indicate the piperacillin MIC. For rows where the exact MIC could not be determined, the MIC was set at >128 μg/mL (black shading) or <0.06 μg/mL (light grey shading).

### Example 26—Minimal Inhibitory Concentration (MIC) Determination of Piperacillin (PIP) in Combination with Zinc Chelator and Tazobactam (TAZ) Vs. Pseudomonas aeruginosa ST773

The effect of the triple combination of the zinc chelator with PIP and TAZ was tested on a clinical isolate of Pseudomonas aeruginosa ST773 carrying genes encoding the β-lactamases NDM-1, OXA-395, PDC-16 (Table 28). Synergy with the zinc chelator was observed at concentrations of 8 μg/mL and 4 μg/mL. The result for the concentration of 16 μg/mL was not interpretable according to EUCAST guidelines.

Experimental results from MIC determination with a triple combination of PIP, TAZ and the zinc chelator are given in Table 28. An X in rows 1-5 in the table indicates growth of the bacterial inoculum, while a blank cell indicates no growth. Concentrations (μg/mL) of PIP are given in row 0. TAZ and the zinc chelator were added in fixed concentrations (TAZ at 16 μg/mL in rows 2-5, zinc chelator at 16 μg/mL in row 3, at 8 μg/mL in row 4, and at 4 μg/mL in row 5). As a control, the effect of PIP alone was tested (row 1). All wells contained bacterial growth medium (caMHB, 5.6 μM Zn²⁺) and were inoculated with the test bacterium. The grey cells indicate the piperacillin MIC. For rows where the exact MIC could not be determined, the MIC was set at >128 μg/mL (black shading) or <0.06 μg/mL (light grey shading).

### Example 27—Minimal Inhibitory Concentration (MIC) Determination of Piperacillin (PIP) in Combination with Zinc Chelator and Avibactam (AVI) Vs. Pseudomonas aeruginosa ST111

The effect of the triple combination of the zinc chelator with PIP and AVI was tested on a clinical isolate of Pseudomonas aeruginosa ST111 carrying genes encoding the β-lactamases VIM-2, OXA-395, PDC-3 (Table 29). Synergy with the zinc chelator was observed at a concentration of 16 μg/mL.

Experimental results from MIC determination with a triple combination of PIP, AVI and the zinc chelator are given in Table 29. Two technical replicates were performed for each sample. An X in rows 1-8 in the table indicates growth of the bacterial inoculum, while a blank cell indicates no growth. Concentrations (μg/mL) of PIP are given in row 0. AVI and the zinc chelator were added in fixed concentrations (AVI at 16 μg/mL in rows 3-8, zinc chelator at 16 μg/mL in rows 5-6 and at 8 μg/mL in rows 7-8). As a control, the effect of PIP alone was tested (rows 1-2). All wells contained bacterial growth medium (caMHB, 29.4 μM Zn²⁺) and were inoculated with the test bacterium. The grey cells indicate the piperacillin MIC.

### Example 28—Minimal Inhibitory Concentration (MIC) Determination of Piperacillin (PIP) in Combination with Zinc Chelator and Tazobactam (TAZ) Vs. Pseudomonas aeruginosa ST111

The effect of the triple combination of the zinc chelator with PIP and TAZ was tested on a clinical isolate of Pseudomonas aeruginosa ST111 carrying genes encoding the β-lactamases VIM-2, OXA-395, PDC-3 (Table 30). Synergy with the zinc chelator was observed at concentrations of 16 μg/mL and 8 μg/mL.

Experimental results from MIC determination with a triple combination of PIP, TAZ and the zinc chelator are given in Table 30. Two technical replicates were performed for each sample. An X in rows 1-8 in the table indicates growth of the bacterial inoculum, while a blank cell indicates no growth. Concentrations (μg/mL) of PIP are given in row 0. TAZ and the zinc chelator were added in fixed concentrations (TAZ at 16 μg/mL in rows 3-8, zinc chelator at 16 μg/mL in rows 5-6 and at 8 μg/mL in rows 7-8). As a control, the effect of PIP alone was tested (rows 1-2). All wells contained bacterial growth medium (caMHB, 29.4 μM Zn²⁺) and were inoculated with the test bacterium. The grey cells indicate the piperacillin MIC.

### Example 29—Minimal Inhibitory Concentration (MIC) Determination of Piperacillin (PIP) in Combination with Zinc Chelator and Avibactam (AVI) Vs. Pseudomonas aeruginosa ST111

The effect of the triple combination of the zinc chelator with PIP and AVI was tested on a clinical isolate of Pseudomonas aeruginosa ST111 carrying genes encoding the β-lactamases VIM-2, OXA-395, PDC-3 (Table 31). Synergy with the zinc chelator was observed at concentrations of 16 μg/mL, 8 μg/mL and 4 μg/mL.

Experimental results from MIC determination with a triple combination of PIP, AVI and the zinc chelator are given in Table 31. An X in rows 1-5 in the table indicates growth of the bacterial inoculum, while a blank cell indicates no growth. Concentrations (μg/mL) of PIP are given in row 0. AVI and the zinc chelator were added in fixed concentrations (AVI at 16 μg/mL in rows 2-5, zinc chelator at 16 μg/mL in row 3, at 8 μg/mL in row 4, and at 4 μg/mL in row 5). As a control, the effect of PIP alone was tested (row 1). All wells contained bacterial growth medium (caMHB, 5.6 μM Zn²⁺) and were inoculated with the test bacterium. The grey cells indicate the piperacillin MIC.

### Example 30—Minimal Inhibitory Concentration (MIC) Determination of Piperacillin (PIP) in Combination with Zinc Chelator and Tazobactam (TAZ) Vs. Pseudomonas aeruginosa ST111

The effect of the triple combination of the zinc chelator with PIP and TAZ was tested on a clinical isolate of Pseudomonas aeruginosa ST111 carrying genes encoding the β-lactamases VIM-2, OXA-395, PDC-3 (Table 32). Synergy with the zinc chelator was observed at concentrations of 16 μg/mL, 8 μg/mL and 4 μg/mL.

Experimental results from MIC determination with a triple combination of PIP, TAZ and the zinc chelator are given in Table 32. An X in rows 1-5 in the table indicates growth of the bacterial inoculum, while a blank cell indicates no growth. Concentrations (μg/mL) of PIP are given in row 0. TAZ and the zinc chelator were added in fixed concentrations (TAZ at 16 μg/mL in rows 2-5, zinc chelator at 16 μg/mL in row 3, at 8 μg/mL in row 4, and at 4 μg/mL in row 5). As a control, the effect of PIP alone was tested (row 1). All wells contained bacterial growth medium (caMHB, 5.6 μM Zn²⁺) and were inoculated with the test bacterium. The grey cells indicate the piperacillin MIC.

### Example 31—Minimal Inhibitory Concentration (MIC) Determination of Meropenem (MEM) in Combination with Zinc Chelator

An experiment was conducted to determine the Minimal Inhibitory Concentration (MIC) of meropenem (MEM) in combination with the zinc chelator according to Example 26 of WO 2018/033719 at varying concentrations of zinc in the medium. The medium was a Mueller Hinton broth from Thermo Fischer. The zinc content was measured as 5.6 μM using ICP-MS. The concentration of zinc in the plasma of healthy animals has been determined to be close to this value. In infectious tissue, the zinc concentration is below the limit of detection (see, for example Bilinskaya et al., Am. Soc. For Microbiology (2020), 58, pages 1-8). Zinc sulfate was then added to this broth to obtain three further concentrations: 15.6 μM, 25.6 μM and 35.6 μM of zinc, keeping all other parameters constant.

As a model bacterial strain for this experiment, the MIC of MEM vs. K. pneumoniae K66-45 (NDM-1) was then determined in broth containing the four concentrations of zinc following the protocol given in Example 1. The EUCAST MIC value for MEM alone (EUCAST: standardization organization for MIC values of antibiotics) is 4-8 μM, meaning that if a bacterial strain is inhibited at lower concentrations than the MIC value, the bacterial strain is sensitive(S) to MEM. Correspondingly, if the bacterial strain is not inhibited at MIC or lower, the strain is resistant (R). If the inhibition is within the MIC range 4-8 μM, the resistance is intermediate (I). The results are provided in Table 33:

Surprisingly, as can be observed in column 2, the EUCAST MIC for MEM alone varies from resistant (R) to sensitive(S) when varying the zinc concentration from 35.6 to 5.6 μM. Even more surprisingly, as can be observed in columns 3-6, the zinc chelator at the standard concentration of 16 μM is ineffective against the bacterial strain at the highest concentration of zinc, while this strain is sensitive to the zinc chelator at 4-8 μM when used tested at a concentration 4 μM. This same dependence of MIC values on zinc concentration in the test medium was observed for other triple combinations according to the invention when employing the same zinc chelator.

### Example 32—Clinical Study

A combination therapy as herein described will be used in the empiric treatment of patients having urinary tract, lung, intra-abdominal or blood stream infections and who are at risk of infection by SBL- and/or MBL-producing Enterobacter, Klebsiella, Pseudomonas, Acinetobacter or Escherichia, or in the treatment of patients diagnosed with an infection in which one or more of these bacteria are present. The three drug substances will be administered together or separately to the patient, for example as multiple intravenous doses or as a continuous daily intravenous dose for about 5 to 28 days depending on the severity of the condition. The antibiotic and SBL-inhibitor will be dosed according to conventional clinical practice and combined with an appropriate dose of the chosen Zn chelator to achieve clinical efficacy and safety of the patient. The Zn chelator may be administered intravenously or orally. An example of a triple combination therapy to be administered to patients will comprise the following Zn chelator:

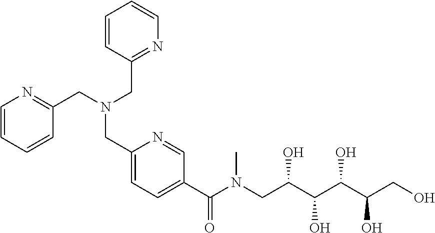 [SMILES: CN(C[C@H](O)[C@@H](O)[C@H](O)[C@H](O)CO)C(=O)c1ccc(CN(Cc2ccccn2)Cc2ccccn2)nc1]

in combination with meropenem (in the form of its sodium salt) and Avibactam (in the form of its sodium salt, optionally Form B of the sodium salt of Avibactam).

1. A method of treatment of a bacterial infection, said method comprising co-administration of an effective amount of each of the following agents to a subject in need thereof:
(A) a selective zinc-chelator which comprises one or more lipophilic, zinc chelating moieties covalently bound to one or more hydrophilic moieties, wherein said zinc chelating moieties are selective for Zn²⁺ ions and wherein said hydrophilic moieties are selected from hydrophilic monomeric, oligomeric and polymeric groups;
(B) a β-lactam antibiotic selected from the group consisting of penicillins, monobactams and carbapenems; and
(C) a serine β-lactamase inhibitor.

2. A method of treatment as claimed in claim 1, wherein agent (A) is a compound of general formula (I), or a stereoisomer, pharmaceutically acceptable salt or prodrug thereof:
$$Q - L - W \qquad ( I )$$
wherein:
Q is a lipophilic, zinc chelating moiety which is selective for Zn²⁺ ions;
L is a covalent bond or a linker; and
W is a hydrophilic monomeric, oligomeric or polymeric group, preferably a hydrophilic group comprising hydrogen bond donor and hydrogen bond acceptor atoms selected from H, N, O, S and P, e.g. a hydrophilic group comprising one or more functional groups selected from —OH, —SH, —CO₂H, —SO₃H, —PO₃H₂, —B(OH)₂, and aliphatic or aromatic nitrogen-containing groups.

3. A method of treatment as claimed in claim 2, wherein the chelating moiety Q comprises one or more optionally substituted heteroaryl groups, preferably two or more heteroaryl groups, e.g. such groups in which each heteroaryl ring has at least one nitrogen atom in the ring structure (e.g. pyridine, especially unsubstituted pyridine).

4. A method of treatment as claimed in claim 3, wherein the chelating moiety Q is derived from picolinic acid and its derivatives (e.g. from picoylamine), preferably wherein the chelating moiety comprises two or more (e.g. two, three or four) 2-pyridyl-methyl units.

5. A method of treatment as claimed in any one of claims 2 to 4, wherein the chelating moiety Q comprises one of the following groups: 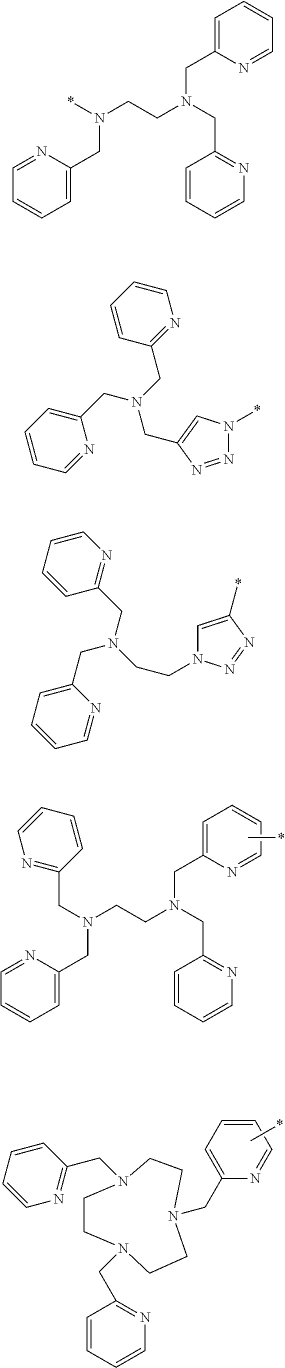 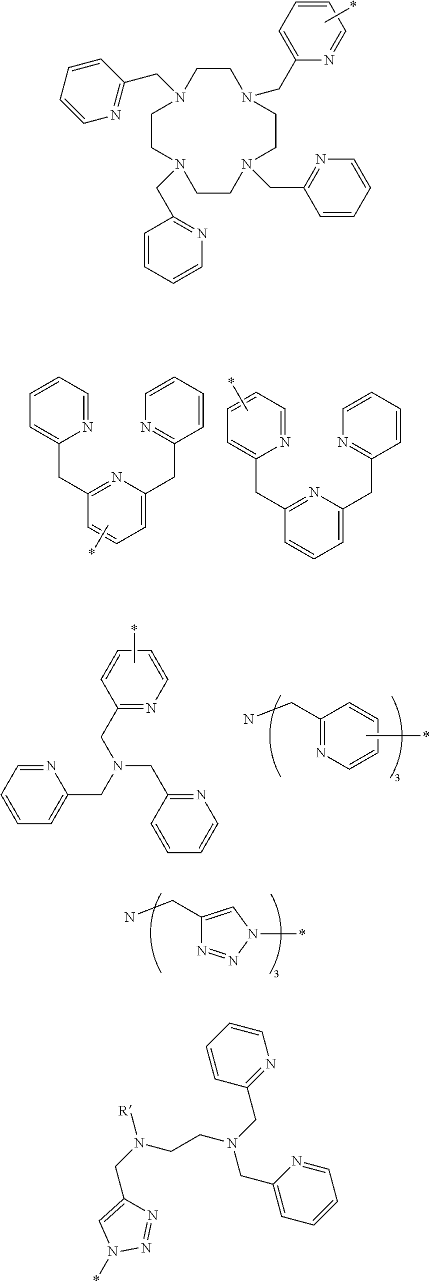 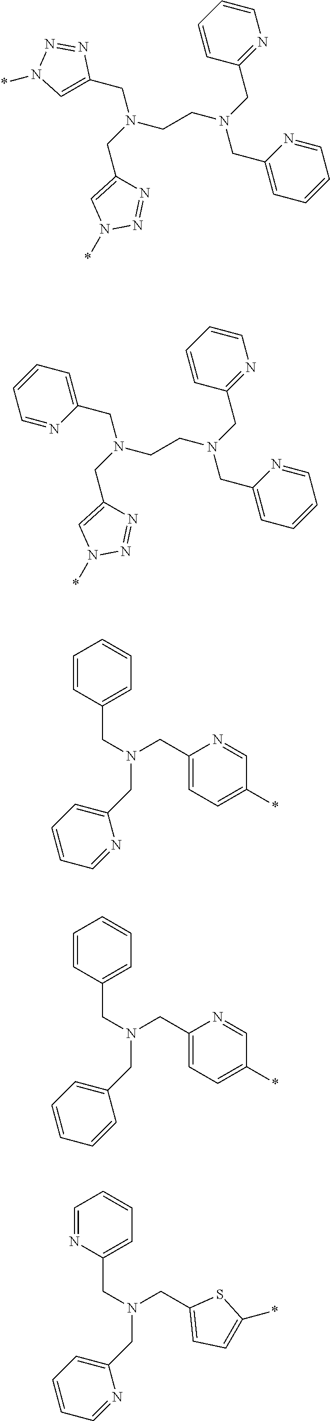 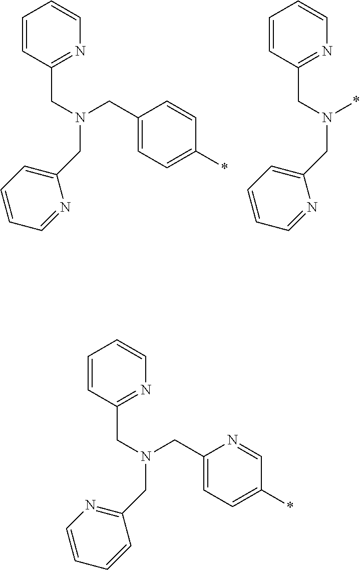
wherein * denotes the point (or points) of attachment of the chelating moiety to the remainder of the molecule, e.g. to the linker group L; and
R′, where present, is H or C₁₋₆ alkyl, e.g. C₁₋₃ alkyl, e.g. methyl.

6. A method of treatment as claimed in any one of claims 2 to 4, wherein the chelating moiety Q comprises the following group:

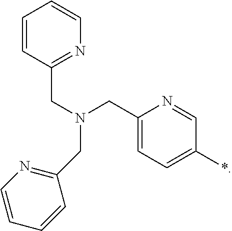

7. A method of treatment as claimed in any one of claims 2 to 6, wherein linker L in formula (I) comprises a bond or an alkylene chain (preferably a C₁₋₈ alkylene, e.g. a C₁₋₆ alkylene) optionally substituted by one or more groups selected from C₁₋₃ alkyl, —O(C₁₋₃ alkyl), and —OR′ (where R′ is H or C₁₋₆ alkyl, preferably C₁₋₃ alkyl, e.g. methyl); and in which one or more —CH₂— groups (e.g. all —CH₂— groups) of the alkylene chain may be replaced by a group independently selected from —O—, —S, —CO—, —NR″— (where R″ is H or C₁₋₆ alkyl, preferably C₁₋₃ alkyl, e.g. methyl), and an optionally substituted carbocyclic or heterocyclic ring (including monocyclic, bicyclic, tricyclic and fused rings; any optional substituents may be selected from C₁₋₆ alkyl, C₁₋₆ alkoxy, halogen, nitro, cyano, amine, and substituted amine).

8. A method of treatment as claimed in any one of claims 2 to 6, wherein linker L in formula (I) comprises a bond, or a C₁₋₈ alkylene chain (preferably a C₁₋₆ alkylene chain, e.g. a C₁₋₃ alkylene chain) in which one or more —CH₂— groups (e.g. all —CH₂— groups) of the alkylene chain are optionally replaced by a group independently selected from —O—, —S—, —CO—, —NR″— (where R″ is independently H or C₁₋₆ alkyl, preferably C₁₋₃ alkyl, e.g. methyl), and an unsubstituted phenyl ring.

9. A method of treatment as claimed in any one of claims 2 to 6, wherein the selective zinc-chelator is a compound of formula (II), or a stereoisomer, pharmaceutically acceptable salt or prodrug thereof: 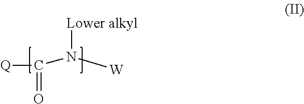
wherein Q and W are as defined in any one of claims 2 to 6; and
“lower alkyl” represents any straight-chained or branched C₁₋₆ alkyl group, preferably a C₁₋₄ alkyl group, e.g. a C₁₋₃ alkyl group such as methyl.

10. A method of treatment as claimed in any one of claims 2 to 9, wherein in formula (I) or formula (II), group W is a polyhydroxylated aliphatic or alicyclic group.

11. A method of treatment as claimed in any one of claims 2 to 9, wherein in formula (I) or formula (II), group W comprises one or more of the following groups: a sugar moiety, a carboxylic acid or derivative thereof (e.g. an ester), an alcohol, an amine or substituted derivative thereof, or a boronic acid.

12. A method of treatment as claimed in claim 11, wherein the sugar moiety is a mono-, di- or polysaccharide, an amino sugar, or a derivative thereof (e.g. an acetylated derivative).

13. A method of treatment as claimed in 12, wherein the sugar moiety is a cyclic or acyclic monosaccharide, preferably wherein the sugar moiety is selected from the following groups and their stereoisomers: 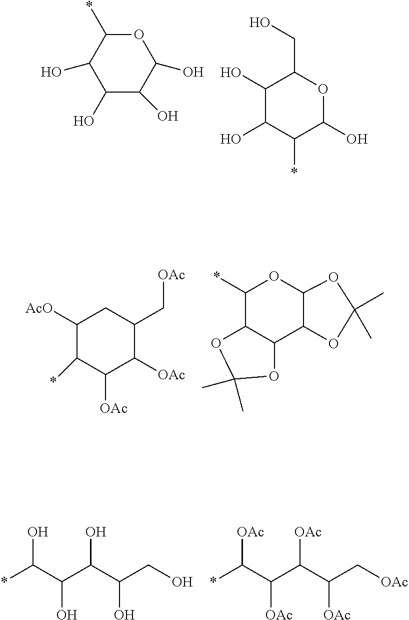
more preferably wherein the sugar moiety is selected from the following groups:

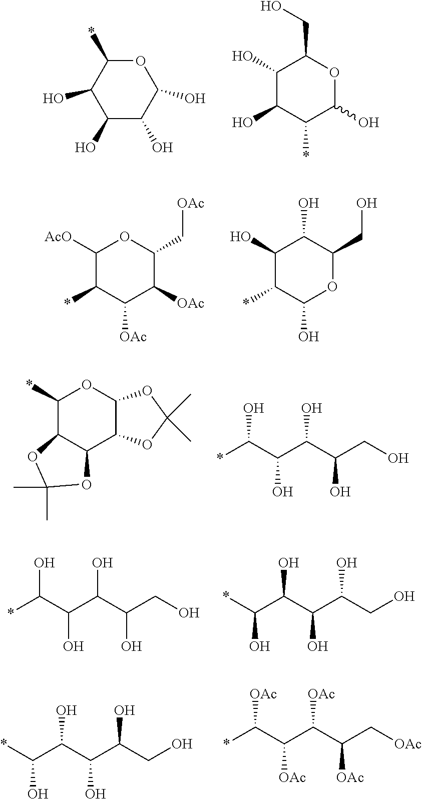

14. A method of treatment as claimed in claim 1, wherein the selective zinc-chelator is selected from the following compounds, their stereoisomers, pharmaceutically acceptable salts and prodrugs:

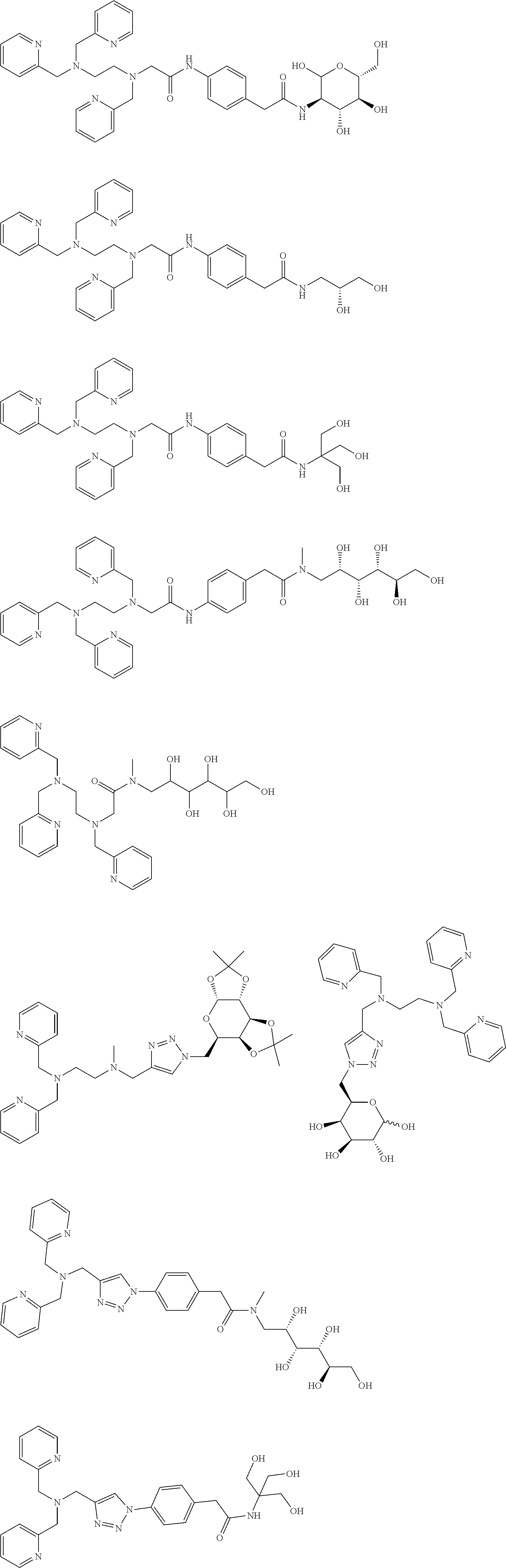

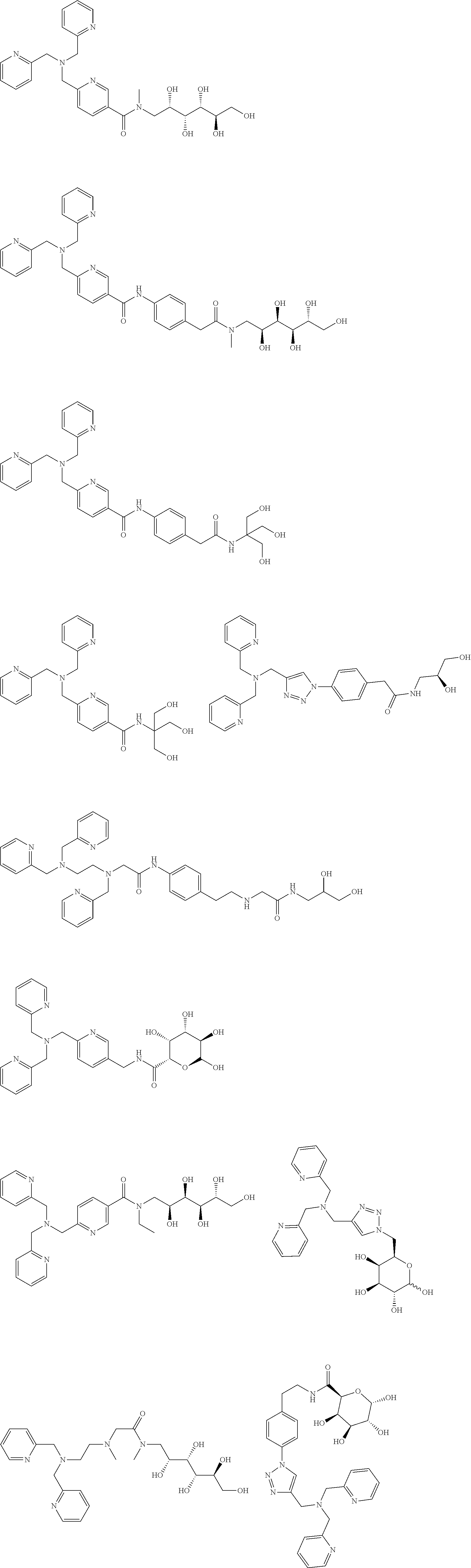

15. A method of treatment as claimed in claim 1, wherein the selective zinc-chelator is a compound having the following structure, or a pharmaceutically acceptable salt, or prodrug thereof.

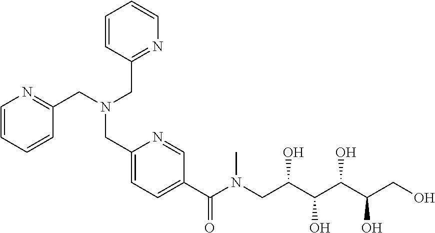 [SMILES: CN(C[C@H](O)[C@@H](O)[C@H](O)[C@H](O)CO)C(=O)c1ccc(CN(Cc2ccccn2)Cc2ccccn2)nc1]

16. A method of treatment as claimed in any one of claims 1 to 15, wherein agent (B) is a penicillin.

17. A method of treatment as claimed in claim 16, wherein the penicillin is selected from the group consisting of cloxacillin, dicloxacillin, flucloxacillin, methicillin, nafcillin, oxacillin, ampicillin, amoxicillin, pivampicillin, bacampicillin, metampicillin, talampicillin, hetacillin, epicillin, phenoxymethylpenicillin, benzylpenicillin, carboxypenicillin, carbenicillin, ticarcillin, temocillin, mezlocillin, piperacillin, azlocillin, and their pharmaceutically acceptable salts, preferably wherein the penicillin is piperacillin, ampicillin, amoxicillin or a pharmaceutically acceptable salt thereof.

18. A method of treatment as claimed in any one of claims 1 to 15, wherein agent (B) is a monobactam.

19. A method of treatment as claimed in claim 18, wherein the monobactam is selected from the group consisting of include aztreonam, aztreonam lysine, tigemonam, nocardicin A, tabtoxin, BAL 30072, SYN 2416 (BAL 19764), carumonam, AIC 499, BOS 228 (LYS 228), MC-1, and their pharmaceutically acceptable salts, preferably wherein the monobactam is aztreonam or a pharmaceutically acceptable salt thereof.

20. A method of treatment as claimed in any one of claims 1 to 15, wherein agent (B) is a carbapenem.

21. A method of treatment as claimed in claim 20, wherein the carbapenem is selected from the group consisting of benapenem, biapenem, doripenem, ertapenem, imipenem, lenapenem, meropenem, panipenem, razupenem, tebipenem, tebipenem, thienpenem, tomopenem and derivatives thereof, e.g. a pharmaceutically acceptable salt thereof.

22. A method of treatment as claimed in claim 20, wherein the carbapenem is meropenem or a pharmaceutically acceptable salt thereof.

23. A method of treatment as claimed in any one of the preceding claims, wherein agent (C) is a serine β-lactamase inhibitor which inhibits at least one SBL in Ambler class A, for example CepA, KPC-2, IMI-1, SME-1, PC1, TEM-1, TEM-2, TEM-3, TEM-30, TEM-50, SHV-1, SHV-2, SHV-10, CTX-M-15, PER-1, VEB-1, PSE-1, CARB-3, or RTG-4; or a serine β-lactamase inhibitor which inhibits at least one SBL in Ambler class C, for example AmpC, CMY-1, ACT-1, FOX-1, MIR-1, GC1, CMY-10, CMY-19, or CMY-37, or a serine β-lactamase inhibitor which inhibits at least one SBL in Ambler class D, for example OXA-1, OXA-10, OXA-11, OXA-15, OXA-23, OXA-48.

24. A method of treatment as claimed in any one of the preceding claims, wherein the serine β-lactamase inhibitor is a diaza-bicyclo-octanone (DBO) compound, a prodrug or a pharmaceutically acceptable salt thereof.

25. A method of treatment as claimed in claim 24, wherein said serine β-lactamase inhibitor is selected from the following compounds, their pharmaceutically acceptable salts and prodrugs:

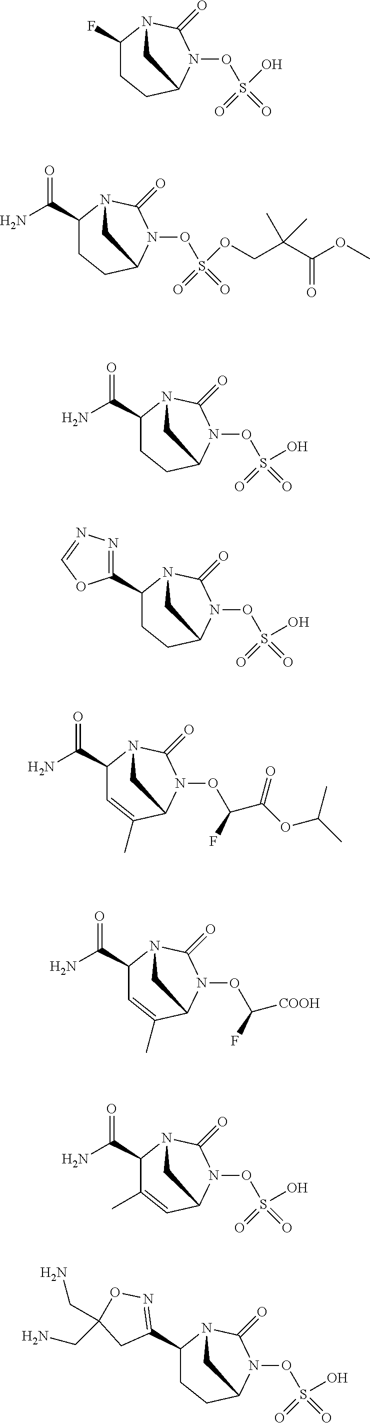

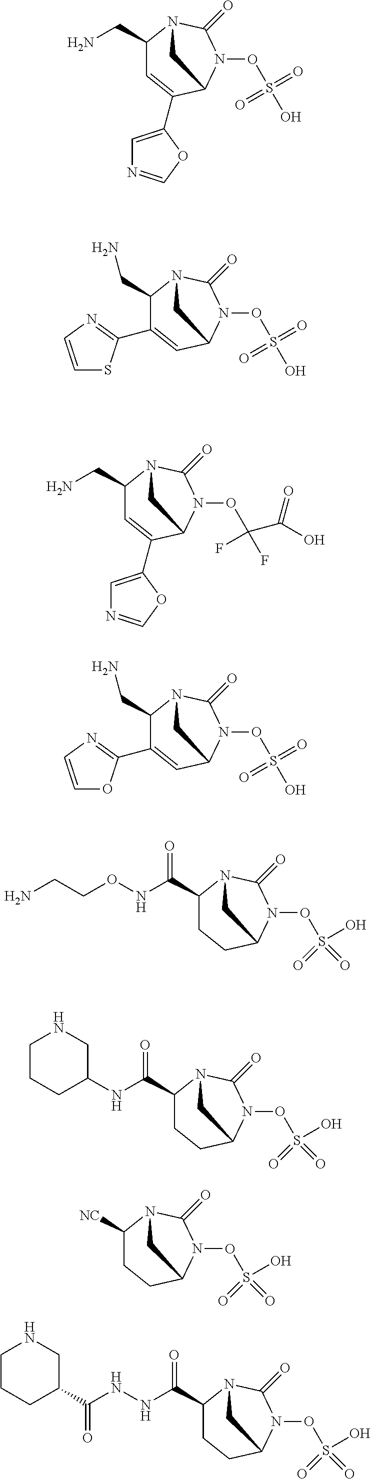

26. A method of treatment as claimed in claim 25, wherein said serine β-lactamase inhibitor is avibactam or a pharmaceutically acceptable salt thereof, preferably the sodium salt of avibactam, for example crystal Form B of the sodium salt of avibactam.

27. A method of treatment as claimed in any one of claims 1 to 23, wherein the serine β-lactamase inhibitor is selected from the group consisting of clavulanic acid, sulbactam, tazobactam, enmetazobactam, their pharmaceutically acceptable salts and prodrugs thereof, preferably wherein the serine β-lactamase inhibitor is sulbactam, tazobactam or a pharmaceutically acceptable salt thereof.

28. A method of treatment as claimed in claim 1, wherein said selective zinc-chelator is a compound as defined in claim 15, said β-lactam antibiotic is meropenem, piperacillin, ampicillin, amoxicillin, aztreonam, or a pharmaceutically acceptable salt thereof, and said serine β-lactamase inhibitor is avibactam, sulbactam, tazobactam or a pharmaceutically acceptable salt thereof.

29. A method of treatment as claimed in claim 1, wherein said selective zinc-chelator is a compound as defined in claim 15, said β-lactam antibiotic is meropenem or a pharmaceutically acceptable salt thereof, and said serine β-lactamase inhibitor is avibactam or a pharmaceutically acceptable salt thereof.

30. A method of treatment as claimed in claim 1, wherein said selective zinc-chelator is a compound as defined in claim 15, said β-lactam antibiotic is meropenem or a pharmaceutically acceptable salt thereof, and said serine β-lactamase inhibitor is sulbactam, tazobactam or a pharmaceutically acceptable salt thereof.

31. A method of treatment as claimed in claim 1, wherein said selective zinc-chelator is a compound as defined in claim 15, said β-lactam antibiotic is aztreonam or a pharmaceutically acceptable salt thereof, and said serine β-lactamase inhibitor is avibactam or a pharmaceutically acceptable salt thereof.

32. A method of treatment as claimed in claim 1, wherein said selective zinc-chelator is a compound as defined in claim 15, said β-lactam antibiotic is piperacillin or a pharmaceutically acceptable salt thereof, and said serine β-lactamase inhibitor is avibactam, tazobactam or a pharmaceutically acceptable salt thereof.

33. A method of treatment as claimed in claim 1, wherein said selective zinc-chelator is a compound as defined in claim 15, said β-lactam antibiotic is ampicillin, amoxicillin or a pharmaceutically acceptable salt thereof, and said serine β-lactamase inhibitor is sulbactam or a pharmaceutically acceptable salt thereof.

34. A method of treatment as claimed in any one of the preceding claims, wherein the bacterial infection is associated with gram-positive or gram-negative bacteria, preferably gram-negative bacteria, more preferably wherein said bacteria are resistant to treatment with one or more antibiotics, particularly bacteria that are resistant to treatment with β-lactam antibiotics.

35. A method of treatment as claimed in claim 34, wherein the bacterial infection is associated with gram-positive or gram-negative bacteria which produce metallo-β-lactamases and/or serine-β-lactamases, preferably gram-negative bacteria which produce serine-β-lactamases.

36. A method of treatment as claimed in any one of the preceding claims, wherein the bacterial infection is caused by a bacteria selected from the group consisting of Acinetobacter baumannii, Acinetobacter calcoaceticus, Acinetobacter haemolyticus, Aeromonas spp, Aeromones hydrophilia, Bacillus cereus Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Bacteroides thetaiotaomicron, Borrelia burgdorferi, Bordetella pertussis, Bordetella parapertussis, Bordetella bronchiseptica, Burkholderia cepacia, Branhamella catarrhalis, Campylobacterfetus, Campylobacter jejuni, Campylobacter coli, Chryseobacterium indologenes Citrobacter freundii, Clostridium difficile, Corynebacterium diphtheriae, Corynebacterium ulcerans, Elizabethkingia meningoseptica, Escherichia coli, Enterobacter cloacae, Enterobacter aerogenes, Enterococcus faecalis, Enterococcus faecium, Francisella tularensis, Gardnerella vaginalis, Haemophilus influenzae, Haemophilus parainfluenzae, Haemophilus haemolyticus, Haemophilus parahaemolyticus, Haemophilus ducreyi, Helicobacter pylori, Legionella pneumophila, Listeria monocytogenes, Kingella, Moraxella, Klebsiella pneumoniae, Klebsiella oxytoca, Legionella pneumophila, Listeria monocytogenes, Morganella morganii, Mycobacterium tuberculosis, Mycobacterium avium, Mycobacterium intracellulare, Moraxella, Mycobacterium leprae, Myroides odoratimimus, Neisseria gonorrhoeae, Neisseria meningitidis Pasteurella multocida, Pasteurella haemolytica, Proteus mirabilis, Proteus vulgaris, Providencia alcalifaciens, Providencia rettgeri, Providencia stuartii, Pseudomonas fluorescens, Pseudomonas aeruginosa, Pseudomonasfluorescens, Pseudomonas acidovorans, Pseudomonas alcaligenes, Pseudomonasputida, Serratia marcescens, Salmonella typhimurium, Salmonella typhi, Salmonella paratyphi, Salmonella enteritidis, Serratia marcescens, Shigella dysenteriae, Shigella flexneri, Shigella sonnei, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, Staphylococcus saccharolyticus. Stenotrophomonas maltophilia, Streptococcus pneumoniae, Streptococcus agalactiae, Streptococcus pyogenes, Vibrio cholerae, Vibrio parahaemolyticus, Yersinia pestis, Yersinia pseudotuberculosis, Yersinia intermedia, Yersinia enterocolitica, and Yersinia pestis.

37. A method of treatment as claimed in claim 36, wherein the bacterial infection is caused by a bacteria selected from the group consisting of Klebsiella pneumoniae, Acinetobacter baumanii, Pseudomonas aeruginosa and Escherichia coli.

38. A method of treatment as claimed in claim 36, wherein the bacterial infection is caused by a bacteria selected from the group consisting of Klebsiella pneumoniae K66-45, Klebsiella pneumoniae ST147, Klebsiella pneumoniae ST101, Klebsiella pneumoniae BAA1705, Acinetobacter baumannii ST15, Acinetobacter baumannii ST25, Pseudomonas aeruginosa ST773 and Pseudomonas aeruginosa ST111.

39. A method of treatment as claimed in claim 36, wherein the bacterial infection is caused by a bacteria selected from the group consisting of Klebsiella pneumoniae Kpn St147, Klebsiella pneumoniae ST101, Klebsiella pneumoniae K66-45 and Klebsiella pneumoniae BAA1705.

40. A method of treatment as claimed in any one of the preceding claims, wherein the subject is a human.

41. A selective zinc-chelator (A) as defined in any one of claims 1 to 15 for use in the treatment of a bacterial infection in a subject by co-administration with (B) a β-lactam antibiotic as defined in any one of claims 1 and 16 to 22; and (C) a serine β-lactamase inhibitor as defined in any one of claims 1 and 23 to 27.

42. A β-lactam antibiotic (B) as defined in any one of claims 1 and 16 to 22 for use in the treatment of a bacterial infection in a subject by co-administration with (A) a selective zinc-chelator as defined in any one of claims 1 to 15; and (C) a serine β-lactamase inhibitor as defined in any one of claims 1 and 23 to 27.

43. A serine β-lactamase inhibitor (C) as defined in any one of claims 1 and 23 to 27 for use in the treatment of a bacterial infection in a subject by co-administration with (A) a selective zinc-chelator as defined in any one of claims 1 to 15; and (B) a β-lactam antibiotic as defined in any one of claims 1 and 16 to 22.

44. A selective zinc-chelator (A), a β-lactam antibiotic (B), or a serine β-lactamase inhibitor (C) for use as claimed in any one of claims 41 to 43, wherein said selective zinc-chelator is a compound as defined in claim 15, said β-lactam antibiotic is meropenem or a pharmaceutically acceptable salt thereof, and said serine β-lactamase inhibitor is avibactam or a pharmaceutically acceptable salt thereof.

45. A selective zinc-chelator (A), a β-lactam antibiotic (B), or a serine β-lactamase inhibitor (C) for use as claimed in any one of claims 41 to 43, wherein said selective zinc-chelator is a compound as defined in claim 15, said β-lactam antibiotic is meropenem or a pharmaceutically acceptable salt thereof, and said serine β-lactamase inhibitor is sulbactam, tazobactam or a pharmaceutically acceptable salt thereof.

46. A selective zinc-chelator (A), a β-lactam antibiotic (B), or a serine β-lactamase inhibitor (C) for use as claimed in any one of claims 41 to 43, wherein said selective zinc-chelator is a compound as defined in claim 15, said β-lactam antibiotic is aztreonam or a pharmaceutically acceptable salt thereof, and said serine β-lactamase inhibitor is avibactam or a pharmaceutically acceptable salt thereof.

47. A selective zinc-chelator (A), a β-lactam antibiotic (B), or a serine β-lactamase inhibitor (C) for use as claimed in any one of claims 41 to 43, wherein said selective zinc-chelator is a compound as defined in claim 15, said β-lactam antibiotic is piperacillin or a pharmaceutically acceptable salt thereof, and said serine β-lactamase inhibitor is avibactam, tazobactam or a pharmaceutically acceptable salt thereof.

48. A selective zinc-chelator (A), a β-lactam antibiotic (B), or a serine β-lactamase inhibitor (C) for use as claimed in any one of claims 41 to 43, wherein said selective zinc-chelator is a compound as defined in claim 15, said β-lactam antibiotic is ampicillin, amoxicillin or a pharmaceutically acceptable salt thereof, and said serine β-lactamase inhibitor is sulbactam or a pharmaceutically acceptable salt thereof.

49. A selective zinc-chelator (A), a β-lactam antibiotic (B), or a serine β-lactamase inhibitor (C) for use as claimed in any one of claims 41 to 48, wherein said bacterial infection is as defined in any one of claims 34 to 39.

50. A pharmaceutical composition comprising:
(A) a selective zinc-chelator as defined in any one of claims 1 to 15;
(B) a β-lactam antibiotic as defined in any one of claims 1 and 16 to 22;
(C) a serine β-lactamase inhibitor as defined in any one of claims 1 and 23 to 27; and
(D) one or more pharmaceutically acceptable carriers or excipients.

51. A pharmaceutical composition as claimed in claim 50, wherein said selective zinc-chelator is a compound as defined in claim 15, said β-lactam antibiotic is meropenem or a pharmaceutically acceptable salt thereof, and said serine β-lactamase inhibitor is avibactam or a pharmaceutically acceptable salt thereof.

52. A pharmaceutical composition as claimed in claim 50, wherein said selective zinc-chelator is a compound as defined in claim 15, said β-lactam antibiotic is meropenem or a pharmaceutically acceptable salt thereof, and said serine β-lactamase inhibitor is sulbactam, tazobactam or a pharmaceutically acceptable salt thereof.

53. A pharmaceutical composition as claimed in claim 50, wherein said selective zinc-chelator is a compound as defined in claim 15, said β-lactam antibiotic is aztreonam or a pharmaceutically acceptable salt thereof, and said serine β-lactamase inhibitor is avibactam or a pharmaceutically acceptable salt thereof.

54. A pharmaceutical composition as claimed in claim 50, wherein said selective zinc-chelator is a compound as defined in claim 15, said β-lactam antibiotic is piperacillin or a pharmaceutically acceptable salt thereof, and said serine β-lactamase inhibitor is avibactam, tazobactam or a pharmaceutically acceptable salt thereof.

55. A pharmaceutical composition as claimed in claim 50, wherein said selective zinc-chelator is a compound as defined in claim 15, said β-lactam antibiotic is ampicillin, amoxicillin or a pharmaceutically acceptable salt thereof, and said serine β-lactamase inhibitor is sulbactam or a pharmaceutically acceptable salt thereof.

56. A pharmaceutical composition as claimed in any one of claims 50 to 55 for use in the treatment of a bacterial infection, preferably for use in the treatment of a bacterial infection as defined in any one of claims 34 to 39.

57. A kit comprising:
(i) a first container containing (A) a selective zinc-chelator as defined in any one of claims 1 to 15;
(ii) a second container containing (B) a β-lactam antibiotic as defined in any one of claims 1 and 16 to 22;
(iii) a third container containing (C) a serine β-lactamase inhibitor as defined in any one of claims 1 and 23 to 27; and
(iv) optionally instructions for carrying out a method of treatment of a bacterial infection in a subject, preferably a bacterial infection as defined in any one of claims 34 to 39.

58. A kit as claimed in claim 57, wherein said selective zinc-chelator is a compound as defined in claim 15, said β-lactam antibiotic is meropenem or a pharmaceutically acceptable salt thereof, and said serine β-lactamase inhibitor is avibactam or a pharmaceutically acceptable salt thereof.

59. A kit as claimed in claim 57, wherein said selective zinc-chelator is a compound as defined in claim 15, said β-lactam antibiotic is meropenem or a pharmaceutically acceptable salt thereof, and said serine β-lactamase inhibitor is sulbactam, tazobactam or a pharmaceutically acceptable salt thereof.

60. A kit as claimed in claim 57, wherein said selective zinc-chelator is a compound as defined in claim 15, said β-lactam antibiotic is aztreonam or a pharmaceutically acceptable salt thereof, and said serine β-lactamase inhibitor is avibactam or a pharmaceutically acceptable salt thereof.

61. A kit as claimed in claim 57, wherein said selective zinc-chelator is a compound as defined in claim 15, said β-lactam antibiotic is piperacillin or a pharmaceutically acceptable salt thereof, and said serine β-lactamase inhibitor is avibactam, tazobactam or a pharmaceutically acceptable salt thereof.

62. A kit as claimed in claim 57, wherein said selective zinc-chelator is a compound as defined in claim 15, said β-lactam antibiotic is ampicillin, amoxicillin or a pharmaceutically acceptable salt thereof, and said serine β-lactamase inhibitor is sulbactam or a pharmaceutically acceptable salt thereof.
